(19) United States
(12) Patent Application Publication
(10) Pub. No.: US 2007/0021365 A1
(43) Pub. Date: Jan. 25, 2007

(54) Inhibition of Lysyl oxidase for treating tumor growth and diagnostics relating thereto

(75) Inventors: Janine Erler, San Francisco, CA (US); Amato Giaccia, Stanford, CA (US)

Correspondence Address: PETERS VERNY JONES & SCHMITT, L.L.P., 425 SHERMAN AVENUE, SUITE 230, PALO ALTO, CA, 94306, US

(73) Assignee: The Board of Trustees of the Leland Stanford Junior University

(21) Appl. No.: 11/471,033

(22) Filed: Jun. 20, 2006

Related U.S. Application Data
(60) Provisional application No. 60/692,435, filed on Jun. 21, 2005.
(60) Provisional application No. 60/795,378, filed on Apr. 27, 2006.

(51) Int. Cl. A61K 48/00; C12Q 1/68; C12Q 1/30; A61K 39/395

(52) U.S. Cl. 514/44; 424/146.1; 435/27; 435/6

(57) ABSTRACT

Disclosed are methods of identifying lysyl oxidase inhibitors and the use of such inhibitors to prevent and treat tumors, particularly metastatic tumors, alone and in combination with chemotherapeutic agents. Further disclosed is the use of lysyl oxidase levels for measuring metastatic potential and survival.

### CROSS-REFERENCE TO RELATED APPLICATIONS

This application claims priority from U.S. Provisional Patent Application No. 60/692,435 filed on Jun. 21, 2005, and from U.S. Provisional Patent Application No. 60/795,378 filed on Apr. 27, 2006, entitled “Inhibition of Lysyl Oxidase for the Prevention of Cancer,” hereby incorporated by reference in their entirety.

### STATEMENT OF GOVERNMENTAL SUPPORT

This invention is supported by Grant No. CA09151 and CA067166 of the National Institutes of Health. The United States government may have certain rights in this invention.

### REFERENCE TO SEQUENCE LISTING, COMPUTER PROGRAM, OR COMPACT DISK

A sequence listing will follow.

### BACKGROUND OF THE INVENTION

1. Field of the Invention

The present invention relates to the field of medicine and particularly to cancer diagnosis and treatment. In particular, the invention relates to lysyl oxidase as an indicator of disease progression and a target for therapeutic agents.

2. Related Art

Background

Lysyl oxidase is an enzyme essential for the formation of the extracellular matrix. Lysyl oxidase catalyzes oxidative deamination of peptidyl lysine and hydroxylysine residues in collagens, and peptidyl lysine residues in elastin. The resulting peptidyl aldehydes spontaneously condense and undergo oxidation reactions to form the lysine-derived covalent cross-links required for the normal structural integrity of the extracellular matrix. In the reaction of lysyl oxidase with its substrates, hydrogen peroxide (H₂O₂) and ammonium are released in quantities stoichiometric with the peptidyl aldehyde product. See, e.g., Kagan et al., J. Cell. Biochem 88:660-72 (2003).

Lysyl oxidase is secreted into the extracellular environment where it is then processed by proteolytic cleavage to a functional 30 kDa enzyme and an 18 kDa propeptide. The 30 kDa lysyl oxidase is enzymatically active whereas the 50 kDa proenzyme is not. Procollagen C-proteinases process pro-lysyl oxidase to its active form and are products of the Bmpl, Tll1 and Tll2 genes. The localization of the enzyme is mainly extracellular, although processed lysyl oxidase also localizes intracellularly and nuclearly. Sequence coding for the propeptide is moderately (60-70%) conserved, whereas the sequence coding for the C-terminal 30 kDa region of the proenzyme in which the active site is located is highly conserved (approximately 95%). See Kagan et al., J. Cell Biochem. 59:329-38 (1995). LOX is induced by a number of growth factors and steroids such as TGF-β, TNF-α and interferon¹⁷. Four LOX related proteins have been identified that all contain the 205 amino acid LOX catalytic domain in their carboxy terminus¹⁷. These family members show some overlap in structure and function¹⁷. Although the main activity of LOX is the oxidation of specific lysine residues in collagen and elastin outside of the cell, it may also act intracellularly, where it may regulate gene expression^{18,19}. In addition, LOX induces chemotaxis of monocytes, fibroblasts and smooth muscle cells²⁰⁻²².

All solid tumors contain areas of low oxygen tension (hypoxia). Hypoxic cells present a great problem in the treatment of cancer because these cells are highly aggressive, metastatic and resistant to therapy. The underlying mechanisms contributing to these features are poorly understood. Metastasis poses a particular problem in breast cancer because there is no effective treatment for the majority of patients with detectable metastatic breast cancer⁴.

The extracellular matrix (ECM) can have a major influence on tumor cells⁵⁶. Mice exposed to hypoxia exhibit tissue specific increases in lysyl oxidase (LOX) activity, an amine oxidase that plays an essential role in the formation and maintenance of the ECM⁷. A recent microarray study confirmed LOX to be a hypoxia-induced gene in a variety of cell lines⁸. However, a biological role of LOX under hypoxic conditions was not identified. LOX initiates the covalent crosslinking of collagens and elastin in the ECM, increasing insoluble matrix deposition and tensile strength⁹. LOX expression is essential for wound healing and normal connective tissue function, and knock-out mice die soon after parturition due to cardiovascular instability¹⁰. Decreased LOX activity is associated with diseases such as Ehler-Danlos syndrome¹¹⁻¹³. Increased LOX activity contributes to fibrotic and tissue remodeling diseases, such as liver cirrhosis¹⁴⁻¹⁶.

Elevated expression of LOX correlates with increased staging in renal cell cancer²⁷, and increased LOX expression is observed in highly metastatic and/or invasive breast cancer cell lines^{28,29}. In contrast, LOX acts as a tumor suppressor in non-tumorgenic revertants of ras-transformed fibroblasts²³. Loss of LOX is associated with tumorigenesis in several cancer types such as gastric, colon and prostate cancers²⁴⁻²⁶. It would thus seem that LOX's tumor suppressive role depends on cell type and transformation status. The propeptide domain (and not the active enzyme) was recently showed to be responsible for the tumor suppressor activities (Palamakumbara et al, JBC 2004). In breast cancer, increased LOX expression is associated with the early stromal reaction³⁰, and treatment with antisense LOX in this cancer cell type prevents in vitro invasion²⁹.

Additional Patent and Publication Citations

Kirschmann et al. “A Molecular Role for Lysyl Oxidase in Breast Cancer Invasion,” Cancer Research, 62:4478-4483 (2002) (Reference 29) discloses that LOX antisense nucleotides transfected into MDA-MB-231 and Hs578T cells inhibit invasion through a collagen IV/laminin/gelatin matrix in vitro.

Payne et al. “Lysyl Oxidase Regulates Breast Cancer Cell Migration and Adhesion through a Hydrogen Peroxide-Mediated Mechanism,” Cancer Research 65, 11429-11436, Dec. 15, 2005, relates to the above paper and discloses that catalytically active LOX regulates in vitro motility/migration and cell-matrix adhesion formation.

Sandel et al. “Merkel cell carcinoma: does tumor size or depth of invasion correlate with recurrence, metastasis, or patient survival?,” laryngoscope 116(5):791-5 (May 2006) discloses a retrospective study correlating clinical and histological criteria from 46 patients diagnosed with Merkel cell carcinoma (metastatic from of skin cancer). A trend was found comparing tumor size or depth of invasion with local recurrence (P=0.07) but with no correlation to regional recurrence (P=0.93 and P=0.60) or distant metastasis (P=0.16 and P=0.24).

Tubiana, et al. “Natural history of human breast cancer: recent data and clinical implications,” Breast Cancer Res Treat. 1991 August; 18(3): 125-40, discloses that the cases studied exhibited a highly significant correlation between tumor size and the probability of distant metastatic dissemination, but that the capacity for lymphatic spread was, on average, acquired much earlier than the capacity for metastatic spread.

Molnar et al. “Structural and functional diversity of lysyl oxidase and the LOX-like proteins,” Biochimica et Biophysica Acta 1647 (2003) 220-224 discusses the roles of lysyl oxidase (LOX) and four lysyl oxidase-like proteins, LOXL, LOXL2, LOXL3 and LOXL4. It is suggested that LOXL proteins may act as tumor suppressors.

U.S. Pat. No. 4,997,854 to et al., issued Mar. 5, 1991, entitled “Anti-fibrotic agents and methods for inhibiting the activity of lysyl oxidase in-situ using adjacently positioned diamine analogue substrates,” discloses a class of anti-fibrotic agents and methods for their use as effective inhibitor substrate analogues of lysyl oxidase in-situ.

U.S. PGPUB 2004/0248871 to Farjanel, et al., published Dec. 9, 2004, entitled “Use of lysyl oxidase inhibitors for cell culture and tissue engineering,” discloses the use of lysyl oxidase inhibitors in the context of the implementation of in vitro cell culture methods which are capable of being used in tissue therapy, or cell therapy, or in experimental pharmacology, i.e., as a method for maintaining the phenotype of a cell by inhibiting LOX. Also disclosed are polyclonal anti-LOX and anti-LOXL1 antibodies from rabbits.

Erler, et al. “Lysyl oxidase is essential for hypoxia-induced metastasis,” Nature 440, 1222-1226 (27 Apr. 2006) was authored by the present inventors.

Erler et al. “Hypoxia promotes invasion and metastasis of breast cancer cells by increasing lysyl oxidase expression,” by the present inventors, is an abstract published online Jun. 17, 2005.

### BRIEF SUMMARY OF THE INVENTION

The following brief summary is not intended to include all features and aspects of the present invention, nor does it imply that the invention must include all features and aspects discussed in this summary.

The present invention comprises, in one aspect, the modulation of lysyl oxidase (LOX) expression, particularly in hypoxic tumors, to inhibit or reduce tumor growth, including primary tumor growth, but particularly including metastatic tumor growth. This enzyme is an important target for new therapeutic agents against metastatic disease.

Thus, in one aspect, provided herein is a method of prevent or reduce metastatic tumor growth, comprising administering to a subject in need thereof an effective amount of an inhibitor of lysyl oxidase activity; and optionally, a pharmaceutically acceptable carrier, thereby preventing or reducing tumor growth. The inhibitor can be a peptide, an antibody, a pharmacological inhibitor, siRNA, shRNA or antisense nucleic acid. In some embodiments, the inhibitor is a small molecule such as BAPN (β-aminoproprionitrile), which forms a covalent bond with LOX, through amine and nitrile groups. In certain embodiments, the tumor is of a particular type, e.g., a breast tumor, a pancreas tumor, a lung tumor, a cervical tumor, a colon tumor or a head and neck tumor. Data on these exemplary types has been obtained. In particular, the method is useful when the tumor is hypoxic, since this condition increases LOX synthesis.

In another aspect, provided herein is a method for identifying a compound that inhibits metastatic tumor cell growth, comprising contacting lysyl oxidase or a cell expressing lysyl oxidase with a candidate compound; and determining the expression or activity of the lysyl oxidase, whereby the candidate compound that reduces the expression or activity of said lysyl oxidase compared to the expression or activity detected in the absence of the compound is identified as the compound that inhibits metastatic tumor cell growth. In particular embodiments, the compound is contacted with lysyl oxidase or a cell expressing lysyl oxidase under hypoxic conditions.

Further, provided herein is a method for identifying a compound that increases the efficacy of chemotherapeutic agents against metastatic tumors, comprising contacting a cell expressing lysyl oxidase with a candidate compound, wherein the compound inhibits the expression or biological activity of lysyl oxidase; contacting the cell with a chemotherapeutic agent either simultaneously or sequentially; determining the viability, growth, or metastasis of the cell, whereby the candidate compound that reduces the viability, growth, or metastasis of the cell compared to the viability or growth of the cell detected in the absence of the treatment or in the presence of either the compound or the chemotherapeutic agent is identified as the compound that increases the efficacy of chemotherapeutic agents against metastatic tumors.

In yet another aspect, provided herein is a method to increase the efficacy of chemotherapeutic agents, comprising administering to a subject in need thereof an effective amount of an inhibitor of lysyl oxidase activity in combination with at least one chemotherapeutic agent; and optionally, a pharmaceutically acceptable carrier, thereby increasing the efficacy of chemotherapeutic agents.

In one aspect, provided herein is a method to stage tumor growth in a cancer patient, comprising assessing the lysyl oxidase levels in the blood, whereby a high lysyl oxidase level indicates the presence of metastatic tumor growth. Also provided is a prognostic marker for identifying a patient as having or being at risk of having metastatic disease which comprises lysyl oxidase and kits using such a prognostic marker.

Also provided herein is a therapeutic composition for prophylaxis and treatment of metastatic tumor growth, said composition comprising: an effective amount of a therapeutically active portion of a lysyl oxidase inhibitor in a pharmaceutically acceptable inert carrier substance, wherein the amount of the inhibitor is effective in preventing and treating metastatic tumor growth.

In yet another aspect, provided herein is a therapeutic composition for prophylaxis and treatment of metastatic tumor growth, said composition comprising: an effective amount of a therapeutically active portion of a lysyl oxidase inhibitor in a pharmaceutically acceptable carrier and at least one chemotherapeutic agent, wherein the amount of the inhibitor is effective in increasing the efficacy of the chemotherapeutic agent in preventing or treating metastatic tumor growth. The inhibitor and chemotherapeutic agent can be administered in any order at least one or more times.

### BRIEF DESCRIPTION OF THE SEVERAL VIEWS OF THE DRAWING

FIG. 1 shows is a diagram showing a LOX HRE (top, A), results of a luciferase assay (middle, B) and increases in luciferas expression (bottom, C);

FIG. 2 is a graph obtained when cells were incubated for 18 h at 2% oxygen (hypoxia) then treated with actinomycin D. Cells were collected 2, 4 and 6 hours after addition of actinomycin D, and mRNA extracted;

FIG. 3 is a graph showing a comparison of LOX protein expression levels with those of CA-IX (carbonic anhydrase IX, upregulated in hypoxia) in a tissue array study from head and neck cancer patients (n=91). Filled bars, LOX positive, open bars, LOX negative. The association value between LOX staining and CAIX stain, P=0.006;

FIG. 4 is a series of graphs A-D showing Kaplan Meyer plots of survival; (A-B) Kaplan-Meyer plots showing that patients with estrogen receptor (ER)-negative staining breast tumors with high LOX expression levels had statistically significant reduced metastasis-free survival. (A: P=0.009) and overall survival (B: P=0.015) than patients low LOX expression levels; (C-D) Kaplan-Meyer plots showing that head and neck cancer patients whose tumors stained positive for LOX had statistically significant reduced metastasis-free survival (C: P=0.02) and overall survival (D: P=0.046) than patients whose tumors stained negative for LOX;

FIGS. 5A and B shows microscopic quantification of metastases in lungs (A) and livers (B) stained with hematoxylin and eosin. Data are numbers of metastases formed at the end of the six-week experiment per mouse (means±s.e.m.) for the ten step sections, based on three independent experimental repeats. Antibody doses: unlabelled, 20 mg kg⁻¹; asterisk, 4 mg kg⁻¹; two asterisks, 1 mg kg⁻¹. Control, n=10; LOX shRNA, n=10; 4 wk BAPN, n=10; 3 wk BAPN, n=3; 2 wk BAPN, n=3; LOX antibody, n=5; FIGS. 5C and D are graphs showing (A) metastases counted weekly over six weeks for ten sections (n=5 mice). Dark solid line, control lung; dashed line, control liver; solid line with squares, bottom dark dashed line, BAPN liver; (B) LOX fluorescent activity assays: left, in vivo enzymatic assay; right, in vitro enzymatic assay (arrowed point indicates 20 mg kg⁻¹ LOX antibody). All data are plotted as means±s.e.m;

FIG. 6 is a pair of graphs showing in vitro invasion of MDA231 (top, A) and SiHa (bottom, B) cells. Results are representative of three independent experimental repeats (means±s.e.m.). Asterisks indicate significant difference (P<0.01) from control cells (Student's t-test). White background, air; grey background, hypoxia;

FIG. 7 is a graph showing in vitro invasion assays performed using cells treated with LOX antisense or sense phosphorothioate-modified oligonucleotides, or with BAPN, prior to incubation in normoxia (white bars) or anoxia (black bars). Results representative of at least three independent experimental repeats (plotted as mean±standard error).

FIG. 8 is a graph (A) showing fibronectin null mouse embryonic fibroblasts subjected to in vitro invasion assays; untreated invasion was compared with that of cells transfected with LOX antisense oligos (Antisense) or incubated with BAPN, and (B) Adhesion of SiHa cells expressing DSRED to matrix was assessed over time;

FIG. 9 is a pair pf graphs showing numbers of lung foci (left,A) and number of colonies formed (right,B); in A is shown tail vein metastasis assays using LOX shRNA or control MDA231 cells; data are plotted as the mean number of lung foci per mouse standard error for the ten step sections, based on two independent experimental repeats; in B, MDA231 control or LOX shRNA cells were grown in soft agar or in Matrigel for 10 days at normoxic or hypoxic conditions; average number of colonies per 10× field of view are plotted;

FIG. 10 is a Kaplan Meyer plot showing survival of mice injected with cells from human lung cancer cell line A549 engineered to stably express shRNA targeting LOX; and

FIG. 11 is a bar graph showing average number of lung tumors in mice from FIG. 10, three weeks after implantation or BAPN treatment; and the inset is a graph showing tumor growth from subcutaneous implantation of shRNA and control tumor cells.

### DETAILED DESCRIPTION OF THE PREFERRED EMBODIMENT

### I. Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference. The heading provided herein are for convenience only and do not limit the invention in any way.

As used herein, “a” or “an” means “at least one” or “one or more.”

As used herein, the term “antibody” means an isolated or recombinant binding agent that comprises the necessary variable region sequences to specifically bind an antigenic epitope. Therefore, an antibody is any form of antibody or fragment thereof that exhibits the desired biological activity, e.g., binding the specific target antigen. Thus, it is used in the broadest sense and specifically covers monoclonal antibodies (including full length monoclonal antibodies), polyclonal antibodies, human antibodies, humanized antibodies, chimeric antibodies, nanobodies, diabodies, multispecific antibodies (e.g., bispecific antibodies), and antibody fragments including but not limited to scFv, Fab, and Fab₂, so long as they exhibit the desired biological activity. The term “human antibody” therefore refers to antibodies containing sequences of human origin, except for possible non-human CDR regions, and does not imply that the full structure of an Ig molecule be present, only that the antibody has minimal immunogenic effect in a human.

The term “anticancer agent” means a chemical compound, biological agent (e.g., antibody) or electromagnetic radiation (especially, X-rays) that is capable of modulating tumor growth or metastasis. These include, e.g., alkylating agents such as busulfan, coordination metal complexes (such as carboplatin, oxaliplatin, and cisplatin), cyclophosphamide (cytoxan), dacarbazine, ifosfamide, mechlorethamine (mustargen), and melphalan, as well as compounds having two labile methanesulfonate groups that are attached to opposite ends of a four carbon alkyl chain. Other examples are non-steroidal aromatase inhibitors and immunotherapeutic agents, i.e., an antibody or antibody fragment that targets cancer cells that produce proteins associated with malignancy. Exemplary immunotherapeutic agents include Herceptin, which targets HER2 or HER2/neu, which occurs in high numbers in about 25 percent to 30 percent of breast cancers; and anti-CD20, which triggers apoptosis in B cell lymphomas. Additional immunotherapeutic agents include immunotoxins, wherein toxin molecules such as ricin, diphtheria toxin and pseudomonas toxins are conjugated to antibodies that recognize tumor specific antigens. Conjugation can be achieved biochemically or via recombinant DNA methods. Other examples include nitrosurea compounds, which are able to travel to the brain so they are used to treat brain tumors. Also included are antimetabolites, which include 5-fluorouracil, methotrexate, gemcitabine (GEMZAR), cytarabine (Ara-C), and fludarabine, and antitumor antibiotics, such as bleomycin, dactinomycin, daunorubicin, doxorubicin (Adriamycin), and idarubicin. Also included are mitotic inhibitors such as taxanes such as paclitaxel and docetaxel, epothilones, etoposide, vinblastine, vincristine, and vinorelbine. The term “anticancer agent” also includes the chemotherapeutic agents described below under the heading “Combination therapy.” The term anticancer agent also includes treatment with a substance that reduces hypoxia in a cell, when such agent is combined with LOX inhibition. Such a substance may include, e.g., p53. See, e.g., Matoba et al., “p53 Regulates Mitochondrial Respiration,” Science 16 Jun. 2006 312: 1650-1653; published online 24 May 2006, and references cited there. A substance that drives cancer cells towards the respiratory pathway and away from the glycolytic pathway would be used advantageously with a LOX inhibitor insofar as LOX would not be upregulated in this case.

As used herein, “inhibit” or “treat” or “treatment” means a postponement of development of the symptoms associated with uncontrolled metastatic tumor growth and/or a reduction in the severity of such symptoms that will or are expected to develop. The terms further include ameliorating existing uncontrolled or unwanted metastatic tumor growth-related symptoms, preventing additional symptoms, and ameliorating or preventing the underlying metabolic causes of symptoms. Thus, the terms denote that a beneficial result has been conferred on a mammalian subject with a metastasis-associated disease or symptom, or with the potential to develop such metastatic disease or symptom. In particular, the term encompasses administration of a composition that prevents metastatic tumor formation and/or inhibits or kills existing metastatic tumors, with resulting clinical benefit. Thus the term “metastatic tumor growth” means the establishment and growth of metastatic tumors, i.e., tumors that have spread from the site of a primary tumor.

As used herein, the term “lysyl oxidase” refers to an enzyme that catalyzes the following reaction: peptidyl-L-lysyl-peptide+O₂+H₂O→peptidyl-allysyl-peptide+NH₃+H₂O₂LO. Other synonyms for lysyl oxidase (EC 1.4.3.13) include protein-lysine 6-oxidase and protein-L-lysine:oxygen 6-oxidoreductase (deaminating). See, e.g., Harris et al., Biochim. Biophys. Acta 341:332-44 (1974); Rayton et al., J. Biol. Chem. 254:621-26 (1979); Stassen, Biophys. Acta 438:49-60 (1976). A copper-containing quinoprotein with a lysyl adduct of tyrosyl quinone at its active center, LO catalyzes the oxidation of peptidyl lysine to result in the formation of peptidyl alpha-aminoadipic-delta-semialdehyde. Once formed, this semialdehyde can spontaneously condense with neighboring aldehydes or with other lysyl groups to from intra- and interchain cross-links. See, e.g., Rucker et al., Am. J. Clin. Nutr. 67:996S-1002S (1998). “LOX” is the enzyme having an amino acid sequence substantially identical to a polypeptide expressed or translated from one of the following sequences: EMBL/GenBank accessions: M94054; AAA59525.1—mRNA; S45875; AAB23549.1—mRNA; S78694; AAB21243.1—mRNA; AF039291; AAD02130.1—mRNA; BC074820; AAH74820.1—mRNA; BC074872; AAH74872.1—mRNA; M84150; AAA59541.1—Genomic DNA.

In some embodiments, the lysyl oxidase enzyme is a lysyl oxidase like enzyme, as described e.g., by Molnar et al., Biochim Biophys Acta. 1647:220-24 (2003). These enzymes include LOXL1, encoded by mRNA deposited at GenBank/EMBL BC015090; AAH15090.1; LOXL2, encoded by mRNA deposited at GenBank/EMBL U89942; LOXL3, encoded by mRNA deposited at GenBank/EMBL AF282619; AAK51671.1; and LOXL4, encoded by mRNA deposited at GenBank/EMBL AF338441; AAK71934.1.

“Lysyl oxidase” or LOX also encompasses a functional fragment or a derivative that still substantially retains its enzymatic activity catalyzing the deamination of lysyl residues. Typically, a functional fragment or derivative retains at least 50% of its lysyl oxidation activity. Preferably, a functional fragment or derivative retains at least 60%, 70%, 80%, 90%, 95%, 99% or 100% of its lysyl oxidation activity. It is also intended that a lysyl oxidase can include conservative amino acid substitutions that do not substantially alter its activity. Suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity. See, e.g., Watson, et al., Molecular Biology of the Gene, 4th Edition, 1987, The Benjamin/Cummings Pub. Co., p. 224.

The term “metastasis” means the ability of tumor cells to invade host tissues and metastasize to distant, often specific organ sites. As is known, this is the salient feature of lethal tumor growths. Metastasis formation occurs via a complex series of unique interactions between tumor cells and normal host tissues and cells. In the context of the present invention, lysyl oxidase activity is critical in the metastatic growth of tumors, i.e., the growth of metastases, particularly under hypoxic conditions. As hypoxic tumors are also the most aggressive and resistant to traditional chemotherapy, agents modulating lysyl oxidase expression and/or function provide a novel therapy against metastatic tumors, particularly chemo-resistant tumors. “Metastasis” is distinguished from invasion. As described in “Understanding Cancer Series: Cancer,” http://www.cancer.gov/cancertopics/understandingcancer/cancer, invasion refers to the direct migration and penetration by cancer cells into neighboring tissues.

The term “pharmaceutically acceptable carrier” is intended to include formulation used to stabilize, solubilize and otherwise be mixed with active ingredients to be administered to living animals, including humans. This includes any and all solvents, dispersion media, coatings, antibacterial and antifungal agents, isotonic and absorption delaying agents, and the like, compatible with pharmaceutical administration. The use of such media and agents for pharmaceutically active substances is well known in the art. See e.g., Remington: The Science and Practice of Pharmacy 20th Ed. (Lippincott, Williams & Wilkins 2003). Except insofar as any conventional media or agent is incompatible with the active compound, such use in the compositions is contemplated.

As used herein, the terms “small interfering RNA” (“siRNA”) or “short interfering RNAS”) refer to an RNA (or RNA analog) comprising between about 10-50 nucleotides (or nucleotide analogs) which is capable of directing or mediating RNA interference. As used herein, “shRNA” should be distinguished from siRNA. As described in Hannon et al., “Unlocking the potential of the human genome with RNA interference,” Nature 431, 371-378 (16 Sep. 2004), shRNA involves expressing mimics of mRNAs in the form of short hairpin RNAs (shRNAs) from RNA polymerase II or III promoters. shRNAs typically have stems ranging from 19 to 29 nucleotides in length, and with various degrees of structural similarity to natural mRNAs. Because these triggers are encoded by DNA vectors, they can be delivered to cells in any of the innumerable ways that have been devised for delivery of DNA constructs that allow ectopic mRNA expression. These include standard transient transfection, stable transfection and delivery using viruses ranging from retroviruses to adenoviruses. Each shRNA expression construct gives rise to a single siRNA.

Importantly, shRNA differs from siRNA in that shRNA results in a stable form of knockdown. Further, as described in Robbins et al., “Stable expression of shRNAs in human CD34+ progenitor cells can avoid induction of interferon responses to siRNAs in vitro,” Nature Biotechnology 24, 566-571 (2006), immunostimulatory effects can be found with lipid-delivered siRNAs not found in Pol III promoter-expressed shRNAs.

shRNA vectors are commercially available, e.g., from OriGene Technologies, Inc. Origene vectors are based on a HuSH pRS plasmid vector which contains both 5 and 3 LTRs of Moloney murine leukemia virus (MMLV) that flank the puromycin marker and the U6-shRNA expression cassette. Upon transient transfection of the plasmids into a packaging cell line, replication deficient viruses can be obtained and used to infected target cells. A puromycin-N-acetyl transferase gene is located downstream of SV40 early promoter, resulting in the resistance to the selection of antibiotics puromycin. The shRNA expression cassette consists of a 21 bp target gene specific sequence, a 10 bp loop, and another 21 bp reverse complementary sequence, all under human U6 promoter. A termination sequence is located immediately downstream of the second 21 bp reverse complementary sequence to terminate the transcription by RNA pol III. The 21 bp gene-specific sequence is sequence-verified to ensure its match to the target gene.

As used herein, the term “subject” means mammalian subjects. Exemplary subjects include, but are not limited to humans, monkeys, dogs, cats, mice, rats, cows, horses, goats and sheep. In some embodiments, the subject has cancer and can be treated with the agent of the present invention as described below.

As used herein, the term “therapeutically effective amount” or “effective amount” refers to an amount of an inhibitor compound that when administered alone or in combination with an additional therapeutic agent to a cell, tissue, or subject is effective to prevent or ameliorate the tumor-associated disease condition or the progression of the disease. A therapeutically effective dose further refers to that amount of the compound sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously.

The term “substantially identical” means identity between a first amino acid sequence that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 60%, or 65% identity, likely 75% identity, more likely 85%, 90%. 91%, 92%, 93%, 94%, 95%, 96%, 97%, 98% or 99% identity to LOX, LOXL-2 or LOXL-4 are termed sufficiently or substantially identical. In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 60%, or 65% identity, likely 75% identity, more likely 85%, 90%. 91%, 92%, 93%, 94%, 95%, 96%, 97%, 98% or 99% identity to nucleic acid SEQ IDs given herein are termed substantially identical.

### II. Generalized Method and Apparatus

The present invention is next described specifically in terms of the aspects of (A) Identification of lysyl oxidase (LOX) inhibitors, (B) LOX inhibitory compositions, (C) Methods of treatment and diagnosis.

A. Identification of Lysyl Oxidase Inhibitors

Provided herein is a method for identifying a compound that inhibits metastatic tumor cell growth, comprising contacting lysyl oxidase or a cell expressing lysyl oxidase with a candidate compound; and determining the expression or activity of the lysyl oxidase, whereby the candidate compound that reduces the expression or activity of said lysyl oxidase compared to the expression or activity detected in the absence of the compound is identified as the compound that inhibits metastatic tumor cell growth. In particular embodiments, the compound is contacted with lysyl oxidase or a cell expressing lysyl oxidase under hypoxic conditions.

Also provided herein is method for identifying a compound that increases the efficacy of chemotherapeutic agents, comprising contacting lysyl oxidase or a cell expressing lysyl oxidase with a candidate compound; and determining the expression or activity of the lysyl oxidase, whereby the candidate compound that reduces the expression or activity of said lysyl oxidase compared to the expression or activity detected in the absence of the compound is identified as the compound that increases the efficacy of chemotherapeutic agents in inhibiting or reducing metastatic tumor growth.

Any suitable source of lysyl oxidase may be employed as an inhibitor target in the present method. The enzyme can be derived, isolated, or recombinantly produced from any source known in the art, including yeast, microbial, and mammalian, that will permit the generation of a suitable product that can generate a detectable reagent or will be biologically active in a suitable assay. In one embodiment, the lysyl oxidase is of human, bovine, or other mammalian origin. See, e.g., Williams, et al., Anal. Biochem. 113:336 (1985); Kirschmann et al., supra; Cancer Res. 62:4478-83 (2002); LOX may be obtained from Accession No. NP00238 (preprotein sequence); Accession No. NM02317 (DNA sequence). A functional fragment or a derivative of lysyl oxidase that still substantially retains its enzymatic activity catalyzing the oxidation of lysyl oxidase can also be used. The lysyl oxidase enzyme can sometimes be the pre-proprotein, proprotein, the protein, or a biologically active fragment thereof.

The enzymatic activity of lysyl oxidase can be assessed by any suitable method. Exemplary methods of assessing lysyl oxidase activity include that of Trackman et al., Anal. Biochem. 113:336-342 (1981); Kagan, et al., Methods Enzymol. 82A:637-49 (1982); Palamakumbura et al., Anal. Biochem. 300:245-51 (2002); Albini et al., Cancer Res. 47: 3239-45 (1987); Kamath et al, Cancer Res. 61:5933-40 (2001); U.S. Pat. No. 4,997,854; and U.S. Patent Application No. 2004/0248871, cited above. For example, the enzymatic activity of the lysyl oxidase can be assessed by detecting and/or quantitating “lysyl oxidase byproducts,” such as H₂O₂ production; collagen pyridinium residuesammonium production; aldehyde product production; lysyl oxidation, deoxypyridinoline (Dpd)—discussed below. One may also detect and quantitate cellular invasive capacity in vitro; cellular adhesion and growth in vitro; and metastatic growth in vivo. In vivo models include, but are not limited to suitable syngeneic models, human tumor xenograft models, orthotopic models, metastatic models, transgenic models, and gene knockout models. See, e.g., Teicher, Tumors Models in Cancer Research (Humana Press 2001).

Hypoxic conditions can be induced or naturally occurring. Hypoxic areas frequently occur in the interior of solid tumor. Hypoxia can also be induced in vivo, particularly in experimental animal models, using diminution or cessation of arterial blood flow to tumor or the administration of vasoconstrictive compounds. See, e.g., U.S. Pat. No. 5,646,185. Exemplary vasoconstrictive compounds include adrenergic direct and indirect agonists such as norepinephrine, epinephrine, phenylephrine, and cocaine. The presence of a hypoxic region in a solid tumor present in a subject can be observed by a number of methods currently known in the art, including nuclear magnetic resonance (NMR) and oxygen electrode pO₂ histography. Such methods may be used in the context of the present invention (as described below), to identify hypoxic treatment target regions and to guide in administering treatment compositions to such regions. In vitro, hypoxic conditions can be induced using any suitable method. For example, cells can be maintained under anoxic (<0.1% O₂) conditions at 37° C. within an anaerobic chamber or under hypoxic (1 to 2% O₂) conditions at 37° C. within a modular incubator chamber filled with 5% CO₂ and 1 to 2% O₂ balanced with N₂. See, e.g., Erler et al., Mol. Cell. Biol. 24:2875-89 (2004).

The present screening method may also include a step of measuring FAK levels. As described below, FAK (Focal Adhesion Kinase [p125FAK]) is activated as part of the cell motility process. When LOX was inhibited with shRNA, FAK phosphorylation is not increased under hypoxic conditions. This can be detected with an anti-phospho-FAK antibody, as described in Methods. In a compound-screening assay, a secondary step may include the detection of phospho-FAK levels both with and without addition of the test compound. A test compound that inhibits LOX will also reduce levels of phospho-FAK.

A compound is an inhibitor of lysyl oxidase expression or biological activity when the compound reduces the expression or activity or lysyl oxidase relative to that observed in the absence of the compound. In one embodiment, a compound is an inhibitor of lysyl oxidase when the compound reduces the incidence of metastasis relative to the observed in the absence of the compound and, in further testing, inhibits metastatic tumor growth. The tumor inhibition can be quantified using any convenient method of measurement. The incidence of metastasis can be assessed by examining relative dissemination (e.g., number of organ systems involved) and relative tumor burden in these sites. Metastatic growth can be ascertained by microscopic or macroscopic analysis, as appropriate. Tumor metastasis can be reduced by about 10%, 20%, 30%, 40%, 50%, 60%, 70%, 80%, 90%, 95% or greater. In some embodiments, the compound can be assessed relative to other compounds that do not impact lysyl oxidase expression or biological activity. The test compounds can be administered at the time of tumor inoculation, after the establishment of primary tumor growth, or after the establishment of local and/or distant metastases. Single or multiple administration of the test compound can be given using any convenient mode of administration, including but not limited to intravenous, intraperitoneal, intratumoral, subcutaneous, and intradermal.

Compounds which are determined to be effective for the prevention or treatment of metastatic tumors in animals, e.g., dogs and rodents, may also be useful in treatment of tumors in humans. Those skilled in the art of treating tumor in humans will know, based upon the data obtained in animal studies, the dosage and route of administration of the compound to humans. In general, the dosage and route of administration in humans is expected to be similar to that in animals, when adjusted for body surface area.

In one embodiment, lysyl oxidase expression is assessed using promoter analysis. Any convenient system for promoter activity analysis can be employed. Typically, the reporter gene system allows promoter activity to be detected using the lysyl oxidase promoter attached to a reporter molecule such that promoter activity results in the expression of the reporter molecule. See, e.g., Ausubel et al., Current Protocols in Molecular Biology (John Wiley & Sons, current edition) at chapter 9.6. Commonly used reporter molecules include CAT, beta-galactosidase, firefly luciferase, and green fluorescent protein. Vectors are available that contain the reporter gene and mammalian processing signals adjacent to restriction sites into which a promoter of interest can be inserted. Usually, the lysyl oxidase promoter-containing vector is transiently transfected into the target cell. However, it can also be stably expressed as described herein. Lysyl oxidase promoter activity can be induced or constitutive. An exemplary lysyl oxidase promoter sequence is disclosed in Csiszar et al., Mol. Biol. Rep. 23:97-108 (1996). Knowledge of the LOX promoter sequence enables the use of drugs acting on this sequence to inhibit LOX expression. For example, as shown below, HIF is a transcription factor upon which LOX expression depends. The LOX promoter site may be blocked by DNA binding drugs, such as antisense DNA. The lysyl oxidase promoter has both positive and negative cis-acting elements, further enabling the use of activating compounds which bind to a negative acting promoter element. See, Jourdan-Le Saux et al. “Functional analysis of the lysyl oxidase promoter in myofibroblast-like clones of 3T6 fibroblast,” J. Cell Biochem. 1997 February; 64(2):328-41.

Also, LOX may be inhibited by degradation of its mRNA. An approach to this form of gene regulation is described in Wilson et al. “Modulation of LDL receptor mRNA stability by phorbol esters in human liver cell culture models,” Lipid Res. 38, 437-446 (1997).

Any suitable cell expressing lysyl oxidase may be employed with the present methods. As used herein, the term “cell” includes a biological cell (e.g., HeLa). The cell can be human or nonhuman. The cell can be freshly isolated (i.e., primary) or derived from a short term- or long term-established cell line. Exemplary biological cell lines include MDA-MB 231 human breast cancer cells, MDA-MB 435 human breast cancer cells, U-87 MG glioma, SCL1 squamous cell carcinoma cells, CEM, HeLa epithelial carcinoma, and Chinese hamster ovary (CHO) cells. Such cell lines are described, for example, in the Cell Line Catalog of the American Type Culture Collection (ATCC, Rockville, Md.).

A cell can express the lysyl oxidase or its promoter endogenously or exogenously (e.g., as a result of the stable transfer of genes). Endogenous expression by a cell as provided herein can result from constitutive or induced expression of endogenous genes.

Exogenous expression by a cell as provided herein can result from the introduction of the nucleic acid sequences encoding lysyl oxidase or a biologically active fragment thereof, or a lysyl oxidase promoter nucleic acid sequence. Transformation may be achieved using viral vectors, calcium phosphate, DEAE-dextran, electroporation, cationic lipid reagents, or any other convenient technique known in the art. The manner of transformation useful in the present invention is conventional and is exemplified in Current Protocols in Molecular Biology (Ausubel, et al., eds. 2000). Exogenous expression of the lysyl oxidase or its promoter can be transient, stable, or some combination thereof. Exogenous expression of the enzyme can be achieved using constitutive promoters, e.g., SV40, CMV, and the like, and inducible promoters known in the art. Suitable promoters are those that will function in the cell of interest.

The lysyl oxidase enzyme or lysyl oxidase-expressing cell can be contacted with the compound in any suitable manner for any suitable length of time. For tumor regions that are accessible to hypodermic delivery of agent, it may be desirable to inject the inhibitory compounds directly into the hypoxic region. The cells can be contacted with the compound more than once during incubation or treatment. Typically, the dose required for an antibody is in the range of about 1 micro-g/ml to 1000 micro-g/ml, more typically in the range of 100 micro-g/ml to 800 micro-g/ml. The exact dose can be readily determined from in vitro cultures of the cells and exposure of the cell to varying dosages of the compound. Typically, the length of time the cell is contacted with the compound is about 5 minutes, about 15 minutes, about 30 minutes, about 1 hour, about 4 hours, about 12 hours, about 36 hours, about 48 hours to about 3 days, more typically for about 24 hours. For in vitro invasion assays, any suitable matrix may be used. In one embodiment, the matrix is reconstituted basement membrane Matrigel™ matrix (BD Sciences).

B. LOX Inhibitor Compositions

Inhibitor compounds are those molecules that inhibit or reduce lysyl oxidase function activity, preferably to reduce metastatic tumor growth. Such inhibition can occur through direct binding of one or more critical binding residues of lysyl oxidase or through indirect interference including steric hindrance, enzymatic alteration of the lysyl oxidase, inhibition of transcription or translation, destabilization of mRNA transcripts, impaired export, processing, or localization of lysyl oxidases, and the like. As used herein, the term “inhibitor compound” includes both protein and non-protein moieties. In some embodiments, the inhibitors are small molecules. Preferably, the inhibitors are compounds with sufficient specificity to avoid systemic toxicity to collagen-rich tissues.

A variety of different test inhibitory compounds may be identified using the method as provided herein. Test inhibitory compounds can encompass numerous chemical classes. In certain embodiments, they are organic molecules, preferably small organic compounds having a molecular weight of more than 50 and less than about 2,500 daltons. Test inhibitory compounds can comprise functional groups necessary for structural interaction with proteins, particularly hydrogen bonding, and may include at least an amine, carbonyl, hydroxyl or carboxyl group, preferably at least two of the functional chemical groups. The test inhibitory compounds can comprise cyclical carbon or heterocyclic structures and/or aromatic or polyaromatic structures substituted with one or more of the above functional groups. Test inhibitory compounds also include biomolecules like peptides, saccharides, fatty acids, steroids, purines, pyrimidines, derivatives, structural analogs or combinations thereof. Test inhibitory compounds of interest also can include peptide and protein agents, such as antibodies or binding fragments or mimetics thereof, e.g., Fv, F(ab′)₂ and Fab, as described further below.

Test inhibitory compounds also can be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Pro-Drugs

The present LOX inhibitors may also be prepared and administered as prodrugs. As is known, a pro-drug is a derivative of an active drug, often a relatively simple derivative, whose properties are considerably reduced, compared to those of the drug. The pro-drug is converted to the active drug in the region of the intended action, in this case a tumor or site of metastasis.

In the present composition, a LOX inhibitory compound is synthesized as a pro-drug, which is converted by hypoxic conditions to the active inhibitor. Other pro-drug strategies may be used, e.g., conversion to a drug with increased oral availability.

Since LOX expression is increased under hypoxic conditions, the hypoxic conditions may be exploited to cause conversion from the pro-drug to the active LOX enzyme inhibitor. Thus, an anti-LOX pro-drug is targeted for conversion at or in a tumor cell, which is typically hypoxic. Nutrients and oxygen are rapidly depleted in tumors. In response to hypoxia, or the exposure to low levels of oxygen, tissues try to restore homeostasis by regulating cellular metabolism and by inducing angiogenesis. Cryer et al. “Changes in enzyme activities in tissues of rats exposed to hypoxia,” Biochem J. 1973 August; 134(4): 1119-1122 describes a list of enzymes which are modulated under hypoxic conditions. For example, it was reported there that alpha-glycerophosphate dehydrogenase activity increased in the liver under hypoxic conditions. This enzyme is found in other tissues, and is an oxidoreductase acting on the CH—OH group of donors. Therefore, for example, a LOX inhibitor may be designed which exploits this metabolic condition through cleavage of a CH—OH bond. Another potential enzyme mechanism involves glutathione S-transferase. Levels of this enzyme (especially the π-isozyme) are elevated in many solid tumors and the enzyme is overexpressed in many drug-resistant tumors. Another structure that has been used for pro-drugs is the folate group, which targets the drug to cells expressing the folic acid receptor, which is typically upregualted in tumor cells. Other pro-drug strategies are disclosed in Hu, “Prodrugs: Effective Solutions for Solubility, Permeability and Targeting Challenges,” IDrugs 2004 7(8):736-742.

Another example of a LOX inhibitor pro-drug is a drug that is activated in a hypoxic cell by a cytochrome p450 enzyme (CYP). Human cancers, including colon, breast, lung, liver, kidney and prostate, are known to express cytochrome P450 (CYP) isoforms including 3A and 1A subfamily members. An example of this type of pro-drug is AQ4N (banoxantrone), a chemotherapeutic pro-drug that is bioreductively activated by CYP3A. The pro-drug conversion is dependent on NADPH and is inhibited by air or carbon monoxide.

AQ4N is an alkylaminoanthraquinone N-oxide (1,4-bis{[2-(dimethylamino-N-oxide)ethyl]amino}5,8-dihydroxyanthracene-9,10-dione) that is activated through enzymatic reduction, selectively under hypoxic conditions, to the corresponding basic amine (See McFadyen et al., “Cytochrome P450 enzymes: Novel options for cancer therapeutics,” Mol Cancer Ther. 2004; 3:363-371 2004). This biotransformation introduces a cationic charge, which can greatly increase the DNA binding affinity, providing a hypoxia-selective prodrug activation mechanism. Thus, under hypoxic conditions, AQ4N can be reduced to a positively charged stable compound AQ4.

Similarly, a LOX inhibitor such as BAPN (discussed below) acts through an amine group. A LOX inhibitory pro-drug can be prepared which contains a derivative to be reduced to a compound having active amino group. Bioreductive moieties, i.e., that can be converted to an active form in a hypoxic cell, are given in U.S. Pat. No. 5,969,133 to Ono, et al., issued Oct. 19, 1999, entitled “Bioreductive cytotoxic agents,” hereby incorporated by reference.

Small Molecule Inhibitors

Exemplary compounds useful in the present invention include, but are not limited to the compounds such as β-aminoproprionitrile (BAPN), as well as the compounds disclosed in U.S. Pat. No. 4,965,288 to Palfreyman, et al., issued Oct. 23, 1990, entitled “Inhibitors of lysyl oxidase, relating to inhibitors of lysyl oxidase and their use in the treatment of diseases and conditions associated with the abnormal deposition of collagen; U.S. Pat. No. 4,997,854 to Kagan, et al., issued Mar. 5, 1991, entitled “Anti-fibrotic agents and methods for inhibiting the activity of lysyl oxidase in-situ using adjacently positioned diamine analogue substrate,” relating to compounds which inhibit LOX for the treatment of various pathological fibrotic states. Further exemplary inhibitors are described in U.S. Pat. No. 4,943,593 to Palfreyman, et al., issued Jul. 24, 1990, entitled “Inhibitors of lysyl oxidase,” relating to compounds such as 2-isobutyl-3-fluoro-, chloro-, or bromo-allylamine; as well as, e.g., U.S. Pat. No. 5,021,456; U.S. Pat. No. 5,5059,714; U.S. Pat. No. 5,120,764; U.S. Pat. No. 5,182,297; U.S. Pat. No. 5,252,608 (relating to 2-(1-naphthyloxymethyl)-3-fluoroallylamine); and U.S. Patent Application No. 2004/0248871. Exemplary inhibitor compounds also include the primary amines reacting with the carbonyl group of the active site of the lysyl oxidases, and more particularly those which produce, after binding with the carbonyl, a product stabilized by resonance, such as the following primary amines: ethylenediamine, hydrazine, phenylhydrazine, and their derivatives, semicarbazide, and urea derivatives, aminonitriles, such as beta-aminopropionitrile (BAPN), or 2-nitroethylamine, unsaturated or saturated haloamines, such as 2-bromo-ethylamine, 2-chloroethylamine, 2-trifluoroethylamine, 3-bromopropylamine, p-halobenzylamines, selenohomocysteine lactone. In another embodiment, the inhibitor compounds are copper chelating agents, penetrating or not penetrating the cells. Additional exemplary compounds include indirect inhibitors such compounds blocking the aldehyde derivatives originating from the oxidative deamination of the lysyl and hydroxylysyl residues by the lysyl oxidases, such as the thiolamines, in particular D-penicillamine, or its analogues such as 2-amino-5-mercapto-5-methylhexanoic acid, D-2-amino-3-methyl-3-((2-acetamidoethyl)dithio)butanoic acid, p-2-amino-3-methyl-3-((2-aminoethyl)dithio)butanoic acid, sodium-4-((p-1-dimethyl-2-amino-2-carboxyethyl)dithio)butane sulphinate, 2-acetamidoethyl-2-acetamidoethanethiol sulphanate, sodium-4-mercaptobutanesulphinate trihydrate.

Inhibitory Antibodies

Burbelo et al. “Monoclonal antibodies to human lysyl oxidase,” Coll Relat Res. 1986 June; 6(2):153-62 discloses hybridoma antibodies against human lysyl oxidase produced by fusing Sp. 2.0-Ag 14 myeloma cells with spleen cells from mice hyperimmunized with lysyl oxidase isolated from umbilical cords. Other monoclonal antibodies, single chain antibodies, or FAb fragments to lysyl oxidase may be prepared.

Preferably the antibody is directed to the catalytic domain of LOX. This domain, in the C-terminal region, contains the elements required for catalytic activity (the copper binding site, tyrosyl and lysyl residues that contribute to the carbonyl cofactor, and 10 cysteine residues. See, Thomassin et al. “The Pro-regions of lysyl oxidase and lysyl oxidase-like 1 are required for deposition onto elastic fibers,” J Biol Chem. 2005 Dec. 30; 280(52):42848-55 for further details.

In one embodiment, the inhibitory compound is an antibody or a biologically active fragment thereof. Conventional methods can be used to prepare the antibodies. For example, by using a peptide or full length lysyl oxidase protein, polyclonal antisera or monoclonal antibodies can be made using standard methods. A mammal, (e.g., a mouse, hamster, or rabbit) can be immunized with an immunogenic form of the peptide that elicits an antibody response in the mammal. Techniques for conferring immunogenicity on a peptide include conjugation to carriers or other techniques well known in the art. For example, the protein or peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

The antibodies can be generated in cell culture, in phage, or in various animals, including but not limited to cows, rabbits, goats, mice, rats, hamsters, guinea pigs, sheep, dogs, cats, monkeys, chimpanzees, apes. Therefore, the antibody useful in the present methods is typically a mammalian antibody. Phage techniques can be used to isolate an initial antibody or to generate variants with altered specificity or avidity characteristics. Such techniques are routine and well known in the art. In one embodiment, the antibody is produced by recombinant means known in the art. For example, a recombinant antibody can be produced by transfecting a host cell with a vector comprising a DNA sequence encoding the antibody. One or more vectors can be used to transfect the DNA sequence expressing at least one V_{L} and one V_{H} region in the host cell. Exemplary descriptions of recombinant means of antibody generation and production include Delves, Antibody Production: Essential Techniques (Wiley, 1997); Shephard, et al., Monoclonal Antibodies (Oxford University Press, 2000); and Goding, Monoclonal Antibodies: Principles and Practice (Academic Press, 1993). One particular inhibitor antibody is an antibody that is directed against the active site of a lysyl oxidase enzyme.

Inhibitory Peptides

In some embodiments, the inhibitory compound is a peptide or peptidomimetic. Exemplary peptides include fibronectin peptides, tropoelastin peptides, or type I collagen peptides. Methods of making peptidomimetics based upon a known sequence are known in the art. See, e.g., U.S. Pat. Nos. 5,631,280; 5,612,895; and 5,579,250. Use of peptidomimetics can involve the incorporation of a non-amino acid residue with non-amide linkages at a given position. One embodiment of the present invention is a peptidomimetic wherein the compound has a bond, a peptide backbone or an amino acid component replaced with a suitable mimic. Examples of unnatural amino acids which may be suitable amino acid mimics include but are not limited to beta-alanine, L-gamma-amino butyric acid, L-alpha-amino butyric acid, L-alpha-amino isobutyric acid, L-epsilon-amino caproic acid, 7-amino heptanoic acid, L-aspartic acid, L-glutamic acid, N-epsilon-Boc-N-alpha-CBZ-L-lysine, N-epsilon-Boc-N-alpha-Fmoc-L-lysine, L-methionine sulfone, L-norleucine, L-norvaline, N-alpha-Boc-N-delta-CBZ-L-ornithine, N-alpha-Boc-N-alpha-CBZ-L-ornithine, Boc-p-nitro-L-phenylalanine, Boc-hydroxyproline, and Boc-L-thioproline. Suitable peptidomimetics include peptidomimetics of a biologically relevant portion of lysyl oxidase, any protein critical to lysyl oxidase expression or biological activity including upstream and downstream components, and the like.

Nucleic Acid Based Inhibitors, RNAi, or Antisense

In another embodiment, the inhibitory compound is an siRNA molecule. RNA interference or “RNAi” refers to a selective intracellular degradation of RNA. RNA interference is a post-transcriptional, targeted gene-silencing technique that uses double-stranded RNA (dsRNA) to degrade messenger RNA (mRNA) containing the same sequence as the dsRNA. The process occurs when an endogenous ribonuclease cleaves the longer dsRNA into shorter, 21- or 22-nucleotide-long RNAs, termed small interfering RNAs or siRNAs. The smaller RNA segments then mediate the degradation of the target mRNA. Kits for synthesis of RNAi are commercially available. See, e.g., U.S. Application No. 20040203145. Thus, RNAi directed to the expression of lysyl oxidase itself, or any critical upstream or downstream effector for lysyl oxidase expression or function is contemplated.

Sometimes the inhibitor compounds are antisense nucleic acids. Lysyl oxidase antisense can be created and introduced into a cell using routine methods and as disclosed herein. See, e.g., Lichtenstein et al., Antisense Technology: A Practical Approach (Oxford University Press 1998).

Combination Therapy

Any suitable anticancer agent, such as a chemotherapeutic agent, may also be employed in the present methods. In one aspect, this invention features methods for inhibiting the invasiveness and metastasis of tumor cells, by contacting the cells with at least one cytotoxic agent and at least one lysyl oxidase inhibitor. In general, the method includes a step of contacting metastatic tumor cells with an amount of at least one cytotoxic agent and at least one lysyl oxidase inhibitor, which, in combination, is effective to reduce or inhibit the invasiveness or metastatic potential of the cell. The present method can be performed on cells in culture, e.g., in vitro or ex vivo, or can be performed on cells present in a subject, e.g., as part of an in vivo therapeutic protocol. The therapeutic regimen can be carried out on a human or on other animal subjects. The lysyl oxidase inhibitor provided herein can be administered in any order relative to the chemotherapeutic agent. Sometimes, the inhibitor and the agent are administered simultaneously or sequentially. They can be administered at different sites and on different dosage regimens. The enhanced therapeutic effectiveness of the combination therapy of the present invention represents a promising alternative to conventional highly toxic regimens of anticancer agents.

Such chemotherapeutic agents include, but are not limited to antimicrotubule agents, topoisomerase I inhibitors, topoisomerase II inhibitors, antimetabolites, mitotic inhibitors, alkylating agents, intercalating agents, signal transduction inhibitors; anti-hormone agents; pro-apoptotic agents; pro-necrosis agents, cytokines such as interferons, interleukins, and tumor necrosis factors, and radiation. Exemplary cytotoxic agents include: paclitaxel, vincristine, vinblastine, vindesine, vinorelbin, taxotere amsacrine, (e.g., Docetaxel), camptothecin, topotecan, irinotecan hydrochloride (e.g., Camptosar), etoposide, mitoxantrone, daunorubicin, epirubicin, merbarone, piroxantrone hydrochloride, methotrexate, 6-mercaptopurine, 6-thioguanine, fludarabine phosphate, cytarabine (Ara-C), trimetrexate, gemcitabine, acivicin, alanosine, pyrazofurin, N-Phosphoracetyl-L-Asparate=PALA, pentostatin, 5-azacitidine, 5-Aza-2′-deoxycytidine, adenosine arabinoside (Ara-A), idarubicin, teniposide, amsacrine, epirubicin, merebarone, piroxantrone hydrochloride, 5-fluorouracil, methotrexate, 6-mercaptopurine, 6-thioguanine, fludarabine phosphate, cytarabine (Ara-C), trimetrexate, gemcitabine, acivicin, alanosine, pyrazofurin, N-Phosphoracetyl-L-Asparate (PALA), pentostatin, 5-azacitidine, 5-Aza-2′-deoxycytidine, adenosine arabinoside (Ara-A), cladribine, ftorafur, UFT (combination of uracil and ftorafur), 5-fluoro-2′-deoxyuridine, 5-fluorouridine, 5′-deoxy-5-fluorouridine, hydroxyurea, dihydrolenchlorambucil, tiazofurin, cisplatin, carboplatin, oxaliplatin, mitomycin C, BCNU (e.g., Carmustine), melphalan, thiotepa, busulfan, chlorambucil, plicamycin, dacarbazine, ifosfamide phosphate, cyclophosphamide, nitrogen mustard, uracil mustard, pipobroman, 4-ipomeanol, dihydrolenperone, spiromustine, geldenamycin, cytochalasins, depsipeptide, leuprolide (e.g., Lupron), ketoconazole, tamoxifen, goserelin (e.g., Zoladex), flutamide, 4′-cyano-3-(4-fluorophenylsulphonyl)-2-hydroxy-2-methyl-3′-(trifluoromethyl)propionanilide, interferon alpha, interferon beta, interferon gamma, interleukin 2, interleukin 4, interleukin 12, tumor necrosis factors, and radiation. The enhanced effect of the lysyl oxidase inhibitor(s) with chemotherapeutic agents, in addition to improving the efficacy of these chemotherapeutic agents, may allow for the administration of lower doses of these chemotherapeutic agents, thus reducing the induction of side effects in a subject.

Formulations

Therapeutic compositions comprising compounds identified as inhibitors using the disclosed methods are also contemplated. In one embodiment, provided herein is a therapeutic composition for prophylaxis and treatment of metastatic tumor growth, said composition comprising: an effective amount of a therapeutically active portion of an inhibitor in a pharmaceutically acceptable carrier substance; wherein said inhibitor inhibits lysyl oxidase, wherein the amount of the inhibitor is effective in preventing and treating metastatic tumor growth. In another embodiment, provided herein is a therapeutic composition for prophylaxis and treatment of metastatic tumor growth comprising an effective amount of a therapeutically active portion of a lysyl oxidase inhibitor in a pharmaceutically acceptable carrier and at least one chemotherapeutic agent, wherein the amount of the inhibitor is effective in increasing the efficacy of the chemotherapeutic agent in preventing or treating metastatic tumor growth.

Various pharmaceutical compositions and techniques for their preparation and use will be known to those of skill in the art in light of the present disclosure. For a detailed listing of suitable pharmacological compositions and associated administrative techniques one may refer to the detailed teachings herein, which may be further supplemented by texts such as Remington: The Science and Practice of Pharmacy 20th Ed. (Lippincott, Williams & Wilkins 2003).

The compositions further include pharmaceutically acceptable materials, composition or vehicle, such as a liquid or solid filler, diluent, excipient, solvent or encapsulating material, i.e., carriers. These carriers are involved in transporting the subject chemical from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Another aspect of the present invention relates to kits for carrying out the combined administration of the lysyl oxidase with other therapeutic compounds. In one embodiment, the kit comprises a lysyl oxidase inhibitor formulated in a pharmaceutical carrier, and at least one cytotoxic agent, formulated as appropriate, in one or more separate pharmaceutical preparations.

The formulation and delivery methods will generally be adapted according to the site and the disease to be treated. Exemplary formulations include, but are not limited to, those suitable for parenteral administration, e.g., intravenous, intra-arterial, intramuscular, or subcutaneous administration, including formulations encapsulated in micelles, liposomes or drug-release capsules (active agents incorporated within a biocompatible coating designed for slow-release); ingestible formulations; formulations for topical use, such as creams, ointments and gels; and other formulations such as inhalants, aerosols and sprays. The dosage of the compounds of the invention will vary according to the extent and severity of the need for treatment, the activity of the administered composition, the general health of the subject, and other considerations well known to the skilled artisan.

Agents as described herein can be incorporated into pharmaceutical compositions suitable for administration. Such compositions typically comprise the agent and a pharmaceutically acceptable carrier. Supplementary active compounds can also be incorporated into the compositions.

C. Methods of Treatment and Diagnosis

Provided herein is a method to prevent or reduce metastatic tumor growth, comprising administering to a subject in need thereof an effective amount of an inhibitor of lysyl oxidase activity; and optionally, a pharmaceutically acceptable carrier, thereby preventing or reducing tumor growth. A detailed description of suitable compositions for use in the present treatment methods is given above. In particular, the method is useful when the tumor is hypoxic. Hypoxic tumors can be readily identified using routine methods in the art. See, e.g., U.S. Pat. No. 5,674,693. As is shown below, the administration of LOX inhibitors has been found to reduce the size of existing tumors, to prevent metastases, and to reduce the size of (or even eliminate) existing metastases.

The present treatment methods also include a method to increase the efficacy of chemotherapeutic agents, comprising administering to a subject in need thereof an effective amount of an inhibitor of lysyl oxidase activity; and optionally, a pharmaceutically acceptable carrier, thereby increasing the efficacy of chemotherapeutic agents. Also contemplated are methods involving the delivery of LOX inhibitory formulations in combination with radiation therapy. Radiation therapy may be used to treat almost every type of solid tumor, including cancers of the brain, breast, cervix, larynx, lung, pancreas, prostate, skin, spine, stomach, uterus, or soft tissue sarcomas. Radiation can also be used to treat leukemia and lymphoma (cancers of the blood-forming cells and lymphatic system, respectively). Radiation dose to each site depends on a number of factors, including the type of cancer and whether there are tissues and organs nearby that may be damaged by radiation. The radiation will typically delivered as X-rays, where the dosage is dependent on the tissue being treated. Radiopharmaceuticals, also known as radionucleotides, may also be used to treat cancer, including thyroid cancer, cancer that recurs in the chest wall, and pain caused by the spread of cancer to the bone (bone metastases).

The subject treated or diagnosed by the present methods includes a subject having or being at risk of having metastatic tumor growth. Such tumors can be a cancer of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus. Tumors treated by compounds of the present methods include, but are not limited to: neoplasm of the central nervous system: glioblastomamultiforme, astrocytoma, oligodendroglial tumors, ependymal and choroids plexus tumors, pineal tumors, neuronal tumors, medulloblastoma, schwannoma, meningioma, meningeal sarcoma: neoplasm of the eye: basal cell carcinoma, squamous cell carcinoma, melanoma, rhabdomyosarcoma, retinoblastoma; neoplasm of the endocrine glands: pituitary neoplasms, neoplasms of the thyroid, neoplasms of the adrenal cortex, neoplasms of the neuroendocrine system, neoplasms of the gastroenteropancreatic endocrine system, neoplasms of the gonads; neoplasms of the head and neck: head and neck cancer, oral cavity, pharynx, larynx, odontogenic tumors: neoplasms of the thorax: large cell lung carcinoma, small cell lung carcinoma, non-small cell lung carcinoma, neoplasms of the thorax, malignant mesothelioma, thymomas, primary germ cell tumors of the thorax; neoplasms of the alimentary canal: neoplasms of the esophagus, neoplasms of the stomach, neoplasms of the liver, neoplasms of the gallbladder, neoplasms of the exocrine pancreas, neoplasms of the small intestine, vermiform appendix and peritoneum, adenocarcinoma of the colon and rectum, neoplasms of the anus; neoplasms of the genitourinary tract: renal cell carcinoma, neoplasms of the renal pelvis and ureter, neoplasms of the bladder, neoplasms of the urethra, neoplasms of the prostate, neoplasms of the penis, neoplasms of the testis; neoplasms of the female reproductive organs: neoplasms of the vulva and vagina, neoplasms of the cervix, adenocarcinoma of the uterine corpus, ovarian cancer, gynecologic sarcomas; neoplasms of the breast; neoplasms of the skin: basal cell carcinoma, squamous carcinoma, dermatofibrosarcoma, Merkel cell tumor; malignant melanoma; neoplasms of the bone and soft tissue: osteogenic sarcoma, malignant fibrous histiocytoma, chrondrosarcoma, Ewing's sarcoma, primitive neuroectodermal tumor, angiosarcoma; neoplasms of the hematopoietic system: myelodysplastic syndromes, acute myeloid leukemia, chronic myeloid leukemia, acute lymphocytic leukemia, HTLV-1, and T-cell leukemia/lymphoma, chronic lymphocytic leukemia, hairy cell leukemia, Hodgkin's disease, non-Hodgkin's lymphomas, mast cell leukemia; neoplasms of children: acute lymphoblastic leukemia, acute myelocytic leukemias, neuroblastoma, bone tumors, rhabdomyosarcoma, lymphomas, renal and liver tumors. In certain embodiment, the tumor is a breast tumor, a pancreas tumor, a lung tumor, a cervical tumor, a colon tumor or a head and neck tumor.

It should be noted that, although leukemia is a cancer of the blood, it might affect other organs, or, in effect, metastasize. In acute leukemias, the abnormal cells may collect in the central nervous system, the testicles, the skin and any other organ in the body. Because leukemia already involves all of the bone marrow in the body, and in many cases, has spread to other organs such as the liver, spleen, and lymph nodes, the staging of leukemia depends on other information that reflects the patient's outlook for survival. Different staging systems are used for different types of chronic leukemia. Some types do not have any staging system.

Staging of solid tumor cancers is well known. The TNM system is one of the most commonly used staging systems. This system has been accepted by the International Union Against Cancer (UICC) and the American Joint Committee on Cancer (AJCC). Most medical facilities use the TNM system as their main method for cancer reporting. PDQ®, the NCI's comprehensive cancer database, also uses the TNM system.

The TNM system, referred to herein as “staging,” is based on the extent of the tumor, the extent of spread to the lymph nodes, and the presence of metastasis.

As to diagnostic methods, the screening or diagnostic analysis of patient samples can be performed in order to determine LOX levels and, accordingly metastatic aggressiveness of tumors. This analysis may be performed prior to the initiation of treatment using lysyl oxidase-specific therapy to identify tumors having elevated LOX expression or activity. Such diagnosis analysis can be performed using any sample, including but not limited to cells, protein or membrane extracts of cells, biological fluids such as sputum, blood, serum, plasma, or urine, or biological samples such as formalin-fixed or frozen tissue sections employing the antibodies of the present invention. Any suitable method for detection and analysis of lysyl oxidase expression can be employed. As used herein, the term “sample” refers to a sample from a human, animal, or to a research sample, e.g., a cell, tissue, organ, fluid, gas, aerosol, slurry, colloid, or coagulated material. The sample may be tested in vivo, e.g., without removal from the human or animal, or it may be tested in vitro. The sample may be tested after processing, e.g., by histological methods. The term “sample” may also refer to a cell, tissue, organ, or fluid that is freshly taken from a human or animal, or to a cell, tissue, organ, or fluid that is processed or stored.

In one aspect, provided herein is a method to stage tumor growth in a cancer patient, comprising assessing the LOX levels in the blood, whereby a high LOX level indicates the presence of metastatic tumor growth. Measurement of LOX levels may take the form of an immunological assay, which detects the presence of a LOX protein with an antibody to the protein. Such assays for other proteins are well known, and may be adapted to detection of LOX proteins. Immunoassays also can be used in conjunction with laser induced fluorescence (see, for example, Schmalzing and Nashabeh, Electrophoresis 18:2184-93 (1997)); Bao, J. Chromatogr. B. Biomed. Sci. 699:463-80 (1997), each of which is incorporated herein by reference). Liposome immunoassays, such as flow-injection liposome immunoassays and liposome immunosensors, also can be used to determine LOX levels according to a method of the invention (Rongen et al., J. Immunol. Methods 204:105-133 (1997), which is incorporated by reference herein). Immunoassays, such as enzyme-linked immunosorbent assays (ELISAs), can be particularly useful in a method of the invention. A radioimmunoassay also can be useful for determining whether a sample is positive for LOX or for determining the level of LOX A radioimmunoassay using, for example, an iodine-125 labeled secondary antibody, may be used.

In addition, one may measure LOX activity, thus ignoring the amount of inactive enzyme. LOX activity may be measured in a number of ways, using a soluble elastin or soluble collagen with labeled lysine as a substrate. Details of an activity assay are given in Royce et al., “Copper metabolism in mottled mouse mutants. The effect of copper therapy on lysyl oxidase activity in brindled (Mobr) mice,” Biochem J. 1982 Feb. 15; 202(2): 369-371. Especially preferred is a chromogenic assay. One is described in Palamakumbura, et al. “A fluorometric assay for detection of lysyl oxidase enzyme activity in biological samples,” Anal Biochem. 2002 Jan. 15; 300(2):245-51. This method was used in the present work to assess LOX inhibition by BAPN and by anti-LOX antibody in cells in culture (using conditioned media) and in mice (using plasma), in a 96-well format.

In addition to measuring the level of LOX in the blood (or urine), one may measure secondary products of LOX activity. For example, deoxypyridinoline (Dpd) is formed by the enzymatic action of lysyl oxidase on lysine residues. Dpd is released into the circulation as a result of osteoclastic degradation of bone. It cannot be re-used, and is cleared by the kidney and excreted unchanged in the urine. Thus, a test based on the Immunodiagnostic

Systems (IDS) Gamma BCT Dpd assay, using a coated tube RIA using an anti-Dpd monoclonal antibody can be used to measure LOX activity.

The following examples are offered to illustrate but not to limit the invention.

### III. Examples

The examples below demonstrate that LOX increased the metastatic potential of human breast cancer cells and other cancer cells in a hypoxic microenvironment, and that inhibition of LOX resulted in inhibition of metastasis. Without wishing to be bound by any one theory, it has been found that secreted LOX acting on collagen is responsible for the invasive and metastatic properties of hypoxic human breast and cervical cancer cells. This secreted LOX may be found outside the cell and therefore represent a target for antibody or peptide treatment.

Inhibition of LOX expression/activity prevented in vitro invasion and in vivo metastasis, particularly in oxygen-deprived conditions. Mice with orthotopically grown breast cancer tumors treated with a chemical inhibitor of LOX, displayed no metastases. It was also found that LOX has diagnostic applications, including the finding that estrogen receptor negative breast cancer patients with high LOX expressing tumors, had poor distant recurrence-free metastasis and overall survival.

### Example 1

### Human Cervical Cancer Cells and Breast Cancer Cells Show Increased LOX Expression Under Hypoxic Conditions

Incubation of human cervical cancer cells and MDA 435 and MDA 231 human breast cancer cells for 18 h under hypoxic (2% oxygen) or anoxic (0.02% oxygen) conditions resulted in elevated LOX mRiNA levels (up to 9 fold) compared with normoxic samples (20% oxygen), assessed by semi- and fully-quantitative RT-PCR (data not shown). This was found to be dependent on the hypoxia-inducible factor (HIF)-1, which increased both transcript expression and stability under hypoxia.

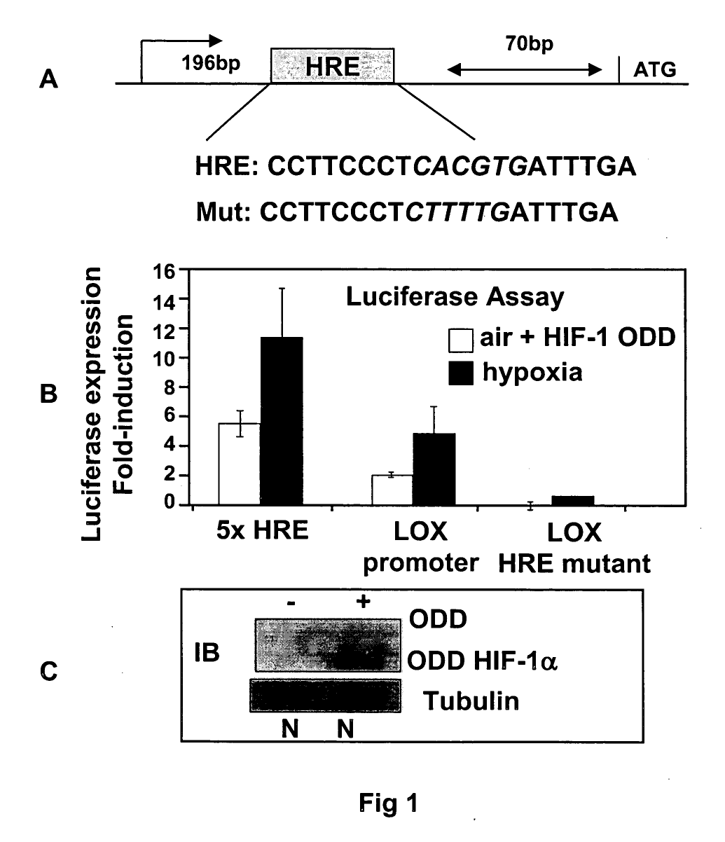

A LOX promoter construct containing up to 1.8 Kb upstream of the LOX translational start site was tested for hypoxic responsiveness employing a standard luciferase assay system. Under normoxic conditions, HIF-1α is rapidly degraded by the proteasome via a mechanism involving the von Hippel Lindau (VHL) ubiquitin E3 ligase¹. Cells lacking VHL express HIF-1α constitutively and thus demonstrate active hypoxic gene expression under aerobic conditions. To assess HIF-1 dependency, LOX mRNA expression levels were analyzed in human RCC4 renal carcinoma cells either lacking VHL or with VHL stably transfected. LOX mRNA levels were induced under hypoxia and anoxia in the VHL expressing cells (VHL+) but were constitutively elevated in the cells lacking VHL (VHL−) even in normoxic conditions. HIF-1-dependency was confirmed in normoxic and MDA 231 cells transfected with non-degradable HIF-1 mutants either lacking the oxygen dependent degradation domain (ODD) domain or mutated at both proline sites required for VHL-mediated degradation (double mutant)³². These cells expressed high levels of HIF-1α protein in air (see lower panel of FIG. 1) as well as elevated LOX mRNA expression that was equivalent to that seen in oxygen deprived cells and cells treated with HIF stabilizing agents, desferrioxamine (DFO) or cobalt chloride (CoCL₂), suggesting HIF-1 induced LOX expression.

### Example 2

### Engineered LOX Promoter Constructs can Modulate Hypoxic Responsiveness

Examination of the human LOX promoter revealed numerous potential hypoxia responsive elements (HREs) to which HIF-1 could bind and regulate gene expression. The LOX promoter sequence was cloned into pGL3-Basic (Promega) and mutated by site-directed mutagenesis (Stratagene), in accordance with the manufacturer's instructions. A positive control containing five hypoxia-responsive elements (HREs) was used. The promoter fragments tested were originally isolated and described by Csiszar et al³³, and show some hypoxia responsiveness in oxygen deprived conditions (FIG. 1 middle panel, black bars). To investigate the role for HIF-1 in transcriptionally regulating LOX, cells were transfected with the ODD mutant construct (see above) such that they expressed high levels of HIF-1α protein in air (lower panel FIG. 1D). Luciferase expression was induced in these aerobic cells when they were transfected with the LOX promoter constructs and the HIF ODD construct, indicating that the LOX promoter was directly responsive to HIF (FIG. 1, white bars). Oxygen deprivation or transfection with HIF-1 resulted in increased luciferase expression, which was abolished upon mutation of an HRE 70 bp upstream of the Start site (FIG. 1). To confirm hypoxic induction of LOX in vivo, mice bearing MDA231 orthotopic tumors were given pimonidazole (100 mg/kg) one hour prior to sacrifice to identify regions of hypoxia. Serial sections of formalin-fixed and paraffin-embedded tumors were stained for LOX and pimo. Staining revealed a strong association between pimo and LOX expression. Taken together, these results indicated that LOX mRNA levels were elevated under hypoxic conditions through a HIF-1-dependent increase in HRE-driven transcript expression.

### Example 3

### Stability of LOX Transcript

The stability of the LOX transcript was examined. Addition of actinomycin D (an inhibitor of transcription) caused a gradual decrease in LOX mRNA levels over time both in air and hypoxia. However, the stability of the LOX transcript was clearly elevated under hypoxic conditions. Ablation of HIF-1α expression by transfection with siRNA dramatically reduced LOX mRNA stability in hypoxia.

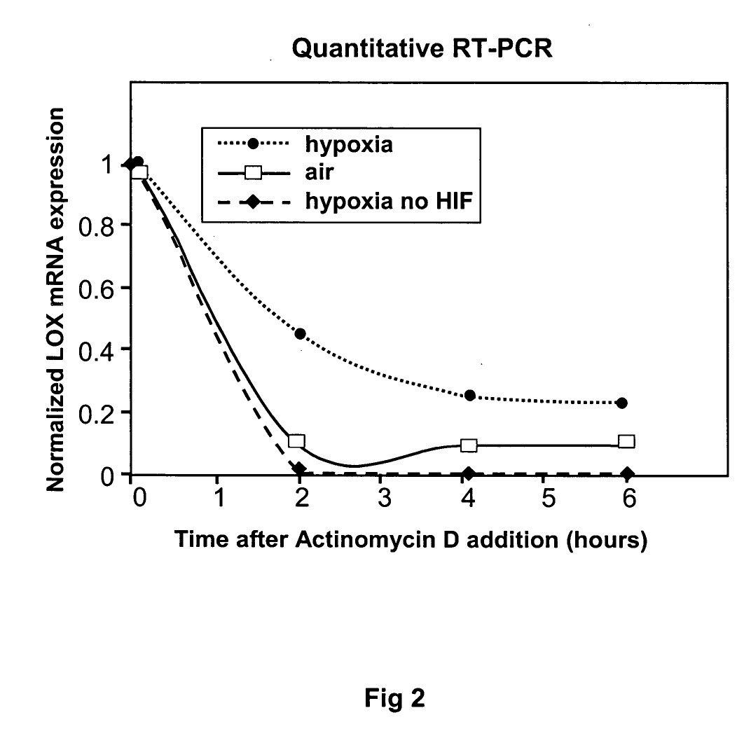

Quantitative RT-PCR was performed to assess LOX mRNA levels, which were normalized to 18S rRNA levels then to LOX mRNA levels at time Oh after actinomycin D addition. As shown in FIG. 2, data was plotted as intra-experimental mean±standard error for triplicate readings. Expression levels were compared to those of cells transfected with HIF-1 alpha targeting siRNA (hypoxia no HIF; previously described⁴⁷ 24 h prior to oxygen deprivation, to investigate HIF-1 involvement. Ablation of HIF-1 alpha protein expression levels was verified by Western Blot. Cells transfected with HIF-1 siRNA (+) or with a scrambled control sequence (−) were incubated under normoxic (N) or hypoxic (H) conditions and immunoblotting performed as described in materials and methods.

### Example 4

### LOX Protein Expression Levels are Elevated in Head and Neck Cancer Tissue

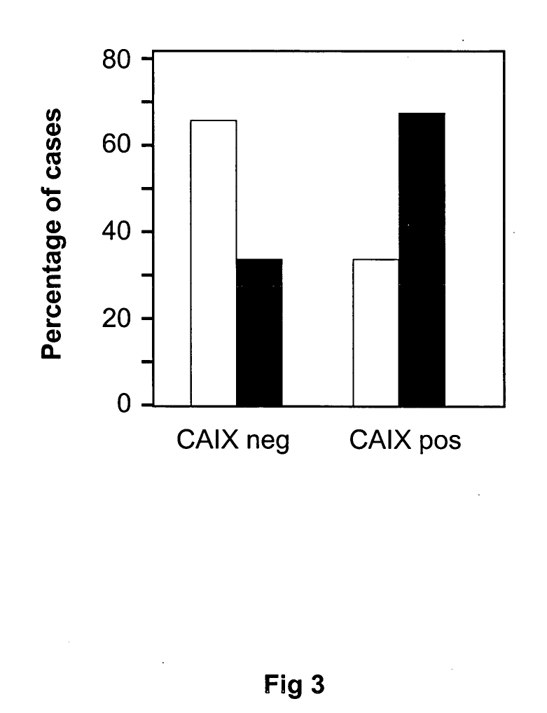

LOX protein expression levels were examined in a head and neck (H&N) cancer patient tissue array through immunohistochemical staining, and were related to tumor hypoxia as determined by carbonic anhydrase-IX (CA-IX) staining (a known intrinsic marker for hypoxia) and patient survival.⁵¹⁻⁵² LOX expression levels significantly correlated with expression of CA-IX (p=0.006). FIG. 3 shows the data from the comparison of LOX protein expression levels with those of CA-IX in a tissue array study from head and neck cancer patients (n=91). Filled bars, LOX positive, open bars, LOX negative. P=0.006. CA IX was significantly increased in LOX negative cases that were CA IX negative.

### Example 5

### LOX Over Expression is Associated with Lower Survival in Estrogen Receptor (ER) Negative Breast Cancer Patients

A recently published micro-array data set from 295 breast cancer patients was additionally analyzed for LOX expression.⁴¹ Estrogen receptor (ER) status was statistically associated with LOX over-expression (higher than the median across the whole data set), and not lymph node status, tumor grade or size, or patient age (Table 1). ER negative breast cancer patients have a worse prognosis, and those at high risk of recurrence currently have no molecularly targeted therapeutic options. LOX over-expression was associated with a statistically significant lower distant metastasis-free survival (p=0.02 FIG. 2E) and overall survival (p=0.015 FIG. 2F) in patients with ER negative tumors, but not in the ER positive tumors or the overall dataset (which mainly comprised of ER positive tumors). The separation seen on these Kaplan-Meyer plots is unlike any previously seen for one gene alone in breast cancer patients. See, Kaplan, E. L.; Meier, Paul. “Nonparametric estimation from incomplete observations.” J. Am. Stat. Assoc. 53, 457-481 (1958). A plot of the Kaplan-Meier estimate of the survival function is a series of horizontal steps of declining magnitude which, when a large enough sample is taken, approaches the true survival function for that population. The value of the survival function between successive distinct sampled observations is assumed to be constant.

TABLE 1
Associations between LOX and clinical criteria in
breast cancer patients⁴¹, determined by Chi Square

| Sample size n = 295 | Probability |
|---|---|
| ER status | p = 0.0014 |
| Lymph node status | p = 0.2679 |
| High Grade (3 v 2 or 1) | p = 0.1117 |
| Size T1-T2 (T1 ≧ 2 cm v T2 > 2 cm) | p = 0.4503 |
| Age (Young < 40 v 40-55 yr) | p = 0.4965 |
| Distant metastasis-free survival | p = 0.1336 |
| Overall survival | p = 0.0674 |
| Distant metastasis-free survival | p = 0.0202 |
| (ER negative only) | |
| Overall survival (ER negative only) | p = 0.0149 |

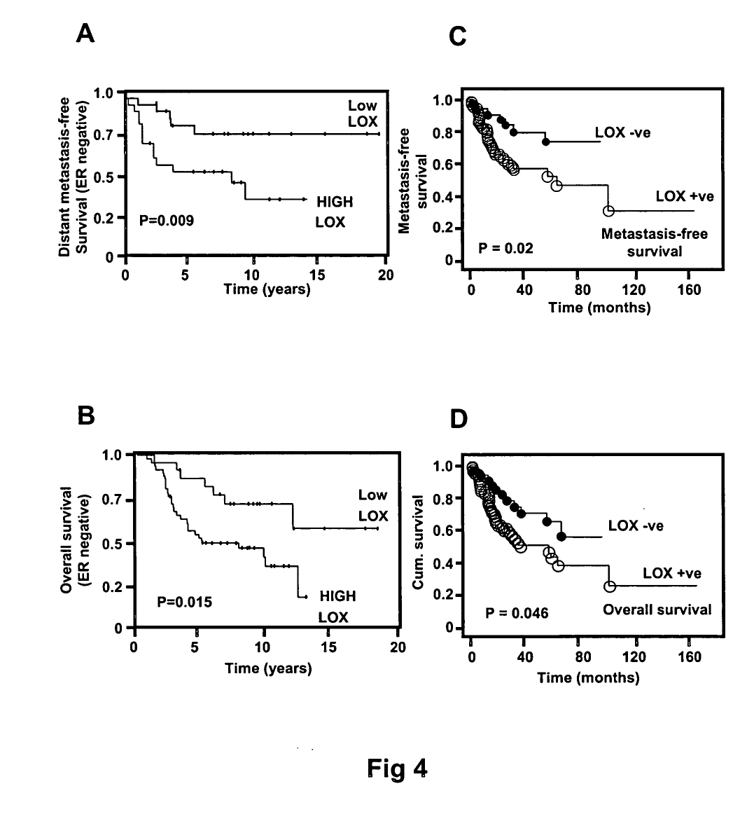

FIGS. 4A and B contains shows Kaplan-Meyer plots showing that patients with ER-negative breast tumors with high LOX expression levels had statistically significant reduced metastasis-free survival. (4A: P=0.009) and overall survival (C: P=0.015) than patients' low LOX expression levels. Patients with high LOX expression had a statistically significant worse outcome with decreased relapse-free survival, metastasis-free survival (p=0.02 FIG. 4C), and overall survival (p=0.046 FIG. 4D).

### Example 6

### In Vivo and In Vitro Growth of shRNA Cells Engineered to Inhibit LOX

How hypoxia-induced LOX influences metastasis and survival in cancer patients was then investigated. MDA 231 human breast cancer cells that stably expressed LOX shRNA through retroviral infection were generated. These cells expressed significantly less LOX mRNA and protein levels particularly under hypoxic conditions compared with cells expressing a scrambled control sequence. LOX mRNA and protein expression levels were examined in cells expressing shRNA specific to LOX (shRNA) or a scrambled control sequence (control), and compared with those of siRNA cells transiently transfected with a mature form of LOX (+LOX). Addition of LOX mRNA restored levels of LOX mRNA expression. In addition, MDA 231 and wild type and LOX shRNA expressing cells were grown in 2D culture and counted each day. Cell numbers increased equally in vitro for shRNA and control cells. Thus, the control and shRNA expressing cells grew at a similar rate in 2D, and had a similar distribution of cells in the different phases of the cell cycle, with the same number of cells traversing S-phase, and demonstrating a slight G1 arrest in response to hypoxia. Tumor size of implanted tumors in vivo was also measured. Tumor size in vivo increased equally in both the control cells and the shRNA cells in vivo for four weeks, after which shRNA cells showed a slight decrease in tumor size while control tumors continued to increase in size (from about 500 to 800 mm³ on average).

### Example 7

### Mice Implanted with Orthotopic Tumors Exhibit Fewer Metastases when LOX Expression is Inhibited by shRNA, with a LOX Antibody, or with BAPN

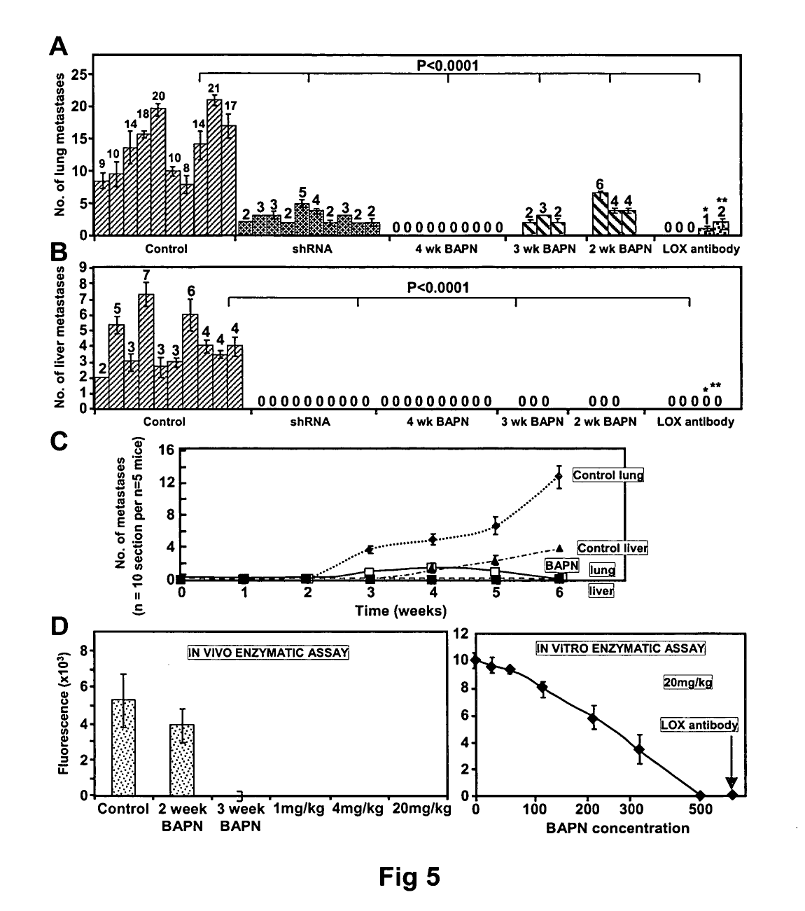

In addition, MDA231 cells were grown as orthotopic tumors in nude mice. Hypoxic regulation of LOX was confirmed in tumors by staining for LOX and pimonidazole. Mice bearing shRNA-expressing tumors had significantly fewer lung metastases and no liver metastases, in contrast with wild-type tumors (FIGS. 5A, B). In FIGS. 5A and B, data were obtained by microscopic quantification of metastases in lungs and livers stained with hematoxylin and eosin. Data shown at the top of each bar are numbers of metastases formed at the end of the six-week experiment per mouse (means±s.e.m.) for the ten step sections, based on three independent experimental repeats. 4 wk BAPN represents treatment started at week 4, etc. The results indicate that treatment started at week 4 actually eliminated tumors that had formed, in view of the number of metastatic tumors found in the control tumors at that point in time.

To evaluate the therapeutic usefulness of inhibiting LOX, mice with control MDA231 tumors were treated with β-aminoproprionitrile (BAPN), a specific and irreversible inhibitor of LOX enzymatic activity. Because BAPN is reported to have effects on other LOX family members (of which there are four, with some overlap in structure and function), mice were also treated with our purified LOX antibody, which cross-reacts with murine LOX, allowing for the assessment of deleterious side effects. Mice treated with BAPN or 20 mg kg⁻¹ antibody did not have any lung metastases or liver metastases (FIGS. 5A,B). Mice that received reduced antibody doses or periods of BAPN treatment displayed significantly fewer lung metastases and no liver metastases, even when metastases had already formed (FIG. 5C). In FIG. 5C, metastases were counted weekly over six weeks for ten sections (n=5 mice). Solid line, with circles control lung; dashed line with triangles, control liver; solid line with squares, BAPN lung; dashed line with squares, BAPN liver. BAPN was started at week 2. Antibody doses: unlabelled, 20 mg kg⁻¹ asterisk, 4 mg kg⁻¹; two asterisks, 1 mg kg⁻¹. Control, n=10; LOX shRNA, n=10; 4 wk BAPN, n=10; 3 wk BAPN, n=3; 2 wk BAPN, n=3; LOX antibody, n=5. Treatment details are given in Methods.

Thus, comparing Examples 6 and 7, inhibition of LOX (with shRNA, BAPN or antibody) had a slight effect on primary tumor growth, but there was no association between tumor size and the number of metastases (data not shown). Reduced LOX activity levels in the blood of BAPN-treated and antibody-treated mice was observed, indicating that the antibody might prevent LOX enzymatic activity, which was confirmed in an in vitro assay (FIG. 5D). On the other hand, the numbers of metastases from tumors formed by highly metastatic MDA 231 cells was dramatically reduced.

### Example 8

### Secreted LOX Plays a Role in Cell Invasion

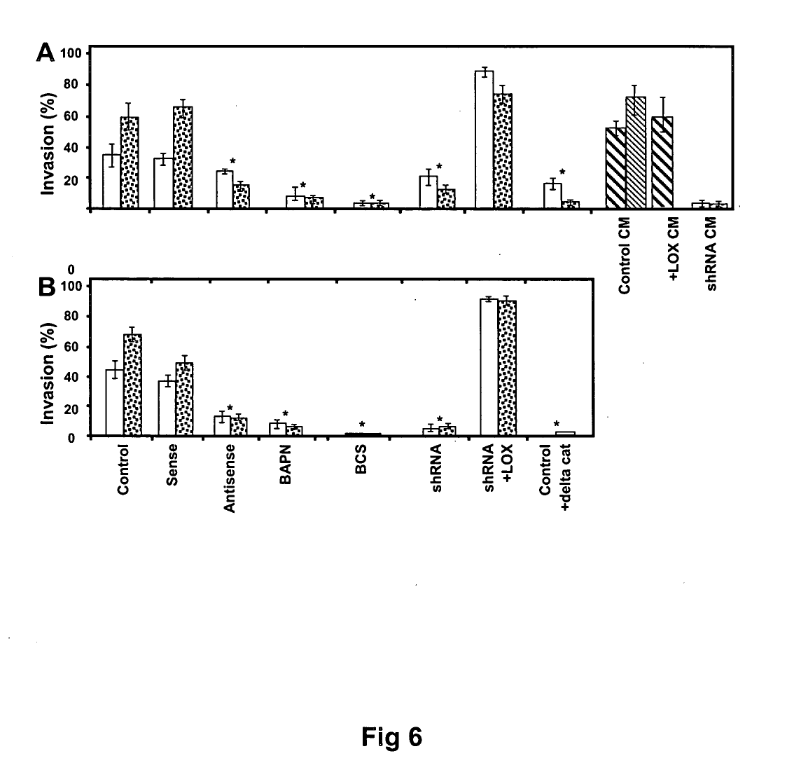

The first step of the metastatic process is cell invasion. Primary tumors from control, BAPN-treated or antibody-treated mice showed evidence of invasion, whereas tumors expressing LOX shRNA did not. The invasion of various human cancer cells in vitro was examined. All cell lines investigated showed significantly increased invasion under hypoxia or anoxia compared with normoxic cells (FIG. 6A, and FIG. 6B). This could be prevented by treatment with LOX antisense oligonucleotides (which decreased LOX mRNA expression; as shown by gels indicating decreased LOX mRNA levels in antisense (AS) treated cells, as verified by RT-PCR by comparing levels to control), BAPN, LOX antibody or shRNA expression, but not with LOX sense oligonucleotides (FIGS. 6A and B). Invasion could be restored in shRNA cells through the overexpression of mature human LOX (which overcame shRNA inhibition) but not with transfection of a catalytically inactive LOX gene (delta cat). Invasion was also increased by the addition of conditioned medium (CM) from control cells or shRNA cells transfected with LOX, but not by the addition of CM from aerobic or hypoxic shRNA cells (FIG. 6). Because LOX is a copper-dependent enzyme that can act intracellularly or extracellularly, cells were treated with bathocuprione disulphonate (BCS), a copper chelator that cannot permeate the cell membrane. This inhibition of extracellular LOX prevented the enhanced invasion of hypoxic cells (FIGS. 6A,B). Neither treatment with BAPN nor treatment with BCS affected cell viability (data not shown), and we observed no differences in histone acetylation in our cell lines with the different treatments, ruling out intracellular LOX involvement (data not shown).

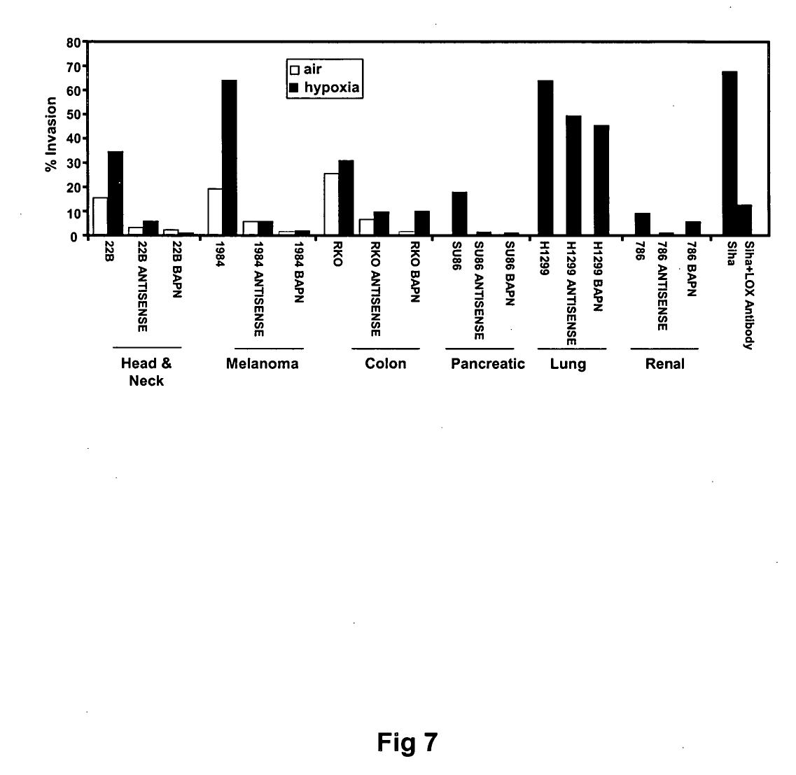

Further data on cell invasion in different types of cancer are presented in FIG. 7. The bars represent in vitro invasion assays using cells treated with LOX antisense or sense phosphorothioate-modified oligonucleotides, or with BAPN, prior to incubation in normoxia (white bars) or anoxia (black bars). Results representative of at least three independent experimental repeats (plotted as mean±standard error).

Taken together, these results demonstrate a role for enzymatically active secreted LOX in the enhanced in vitro invasion observed in oxygen-deprived cells.

The ability of cells to reorganize and contract three-dimensional type I collagen gels is regarded as an in vitro model for invasion. In air, control cells showed a branching morphogenesis typical of invasive human cancer cells grown on collagen, which was enhanced by hypoxia. The LOX shRNA cells grew in a markedly different manner, remaining in spheroid-like cell clusters, showing little branching in air or hypoxia. These results also indicate a role for LOX in the development of an invasive phenotype of hypoxic human cancer cells in vitro.

Acquisition of motility is required before cells can migrate and invade. Invasive cell migration is a multi-step process¹⁷ that commences with pseudopod protrusion at the leading edge driven by actin polymerization, resulting in focal adhesion formation and the activation of integrin and focal adhesion kinase (FAK). Intense immunofluorescent staining of extracellular LOX was observed at the leading edge of MDA231 cells grown on collagen, particularly in hypoxic conditions. LOX protein expression extended along hairlike fibers protruding from the cell surface into the collagen matrix. This was not observed in the LOX shRNA cells, which showed levels of intracellular LOX expression equivalent to the controls containing no antibody. Remodeling of the actin cytoskeleton with increased formation of stress fibers and focal adhesion in MDA231 control cells particularly in hypoxia was observed, which were not seen in cells expressing LOX shRNA. Hypoxia additionally increased FAK phosphorylation (activation) in control cells (consistent with previous reports) but not in the LOX shRNA cells.

### Example 9

### Fibronectin is Not Necessary for LOX Action

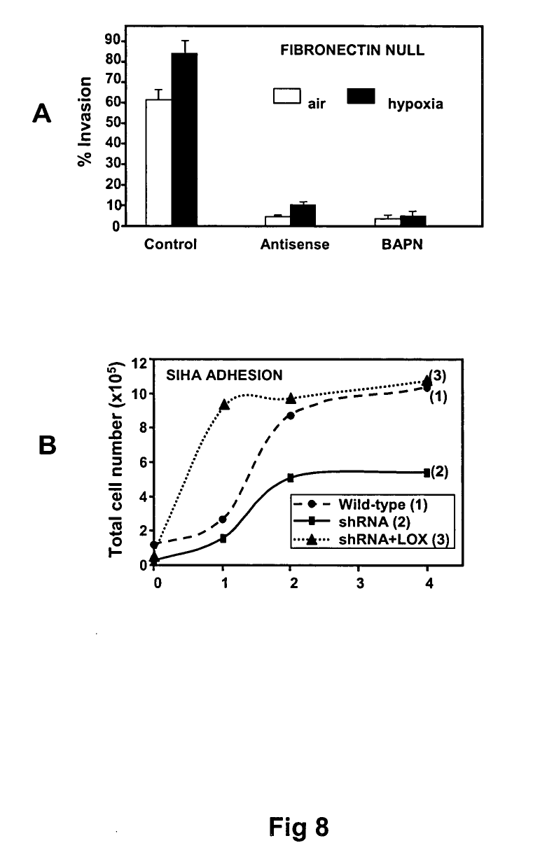

A role for fibronectin (FN) was sought, because it was strongly associated with LOX expression in the breast cancer microarray data set that was analyzed (data not shown), is reported to regulate invasion, bind to integrins and interact with LOX, and is hypoxia induced. However, neither FAK phosphorylation nor cell invasion was affected by transfection with FN antisense oligonucleotides or by the addition of plasma FN (pFN) or cellular FN (cFN) (which do and do not interact with LOX, respectively). Moreover, FAK phosphorylation was intact in FN-null cells but was decreased by transfection with LOX antisense oligonucleotides, which strongly inhibited invasion (similarly to BAPN addition). FAK phosphorylation could be induced in the cells expressing LOX shRNA by transfection with mature LOX (but not catalytically dead LOX), confirming a role for LOX in FAK phosphorylation. This could be prevented by the addition of BAPN or catalase (consistent with a recent report⁹), or a blocking antibody against b1 integrin but not against a6 integrin. These results show an enzymatic role for LOX in the regulation of FAK through b1 integrin, in a FN-independent manner. It is proposed here that this is because LOX increases fibrillar collagen, which is a ligand for b1 integrin. Other integrin pathways have been reported to mediate hypoxia-induced invasion. Complete elimination of FAK activity in hypoxic control cells was achieved when both β1 integrin activation and hydrogen peroxide production (a by-product of LOX activity) were prevented (FIG. 8A), demonstrating the importance of both these mechanisms in hypoxia.

### Example 10

### LOX Increases Adhesion to Collagen and Matrigel (rtm) Cell Substrates

Increased adhesion is a characteristic of invasive cells with a mesenchymal phenotype and is essential for their motility. Both the MDA231 and cells expressing LOX shRNA showed decreased adhesion to collagen I (FIG. 8B) and Matrigel (data not shown), which could be restored on transfection with mature LOX. In invasive migration, increased adhesion additionally results in the recruitment of proteases (such as matrix metalloproteinases (MMPs)) to the attachment sites. Although expression levels of MMP-2 and MMP-9 were elevated by hypoxia, consistent with previous reports21, and MMP-2 and MMP-14 expression was strongly correlated with LOX in breast cancer patients, LOX expression did not affect MMP activities (data not shown). However, time-lapse photography of wild-type cells and cells expressing LOX shRNA within a collagen matrix or subjected to scratch assays revealed that LOX inhibition completely prevented cell movement. Taken together, these results demonstrate a crucial role for LOX in cell motility through its effects on cell adhesion through FAK activation.

### Example 11

### LOX Inhibition by shRNA Results in Fewer Metastases in a Tail Vein Assay

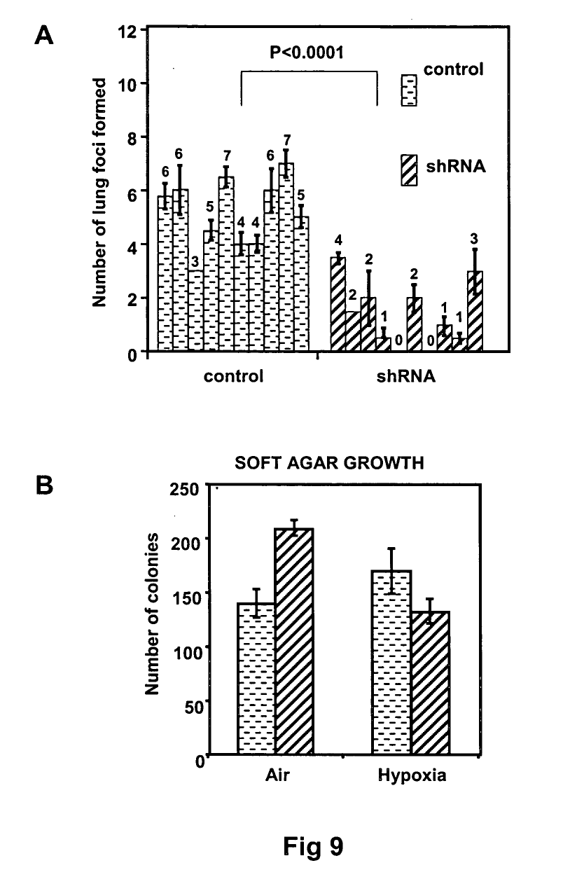

Elimination of the early invasive steps of metastasis through tail-vein studies revealed a role for LOX in the later stages of metastasis: mice injected with LOX shRNA expressing MDA 231 breast cancer cells had fewer lung foci (FIG. 9A). Although the cloning efficiency of the cells was similar to that of control cells, their metastatic growth in vitro was severely impaired (FIG. 9B). This seemed to be due to defective cell-matrix or extracellular matrix (ECM)-protein interactions required to permit and support abundant growth. Consistent with this is our observation that the metastatic lesions formed in our orthotopic studies were much larger in mice implanted with control cells than with shRNA cells, and were completely absent in BAPN treated mice. Close examination of these metastatic lesions revealed they were mostly composed of inflammatory cells.

### Example 12

### Mice Bearing Tumors Expressing LOX shRNA Survive Beyond those with Non-Treated Tumors and Exhibited No Metastases

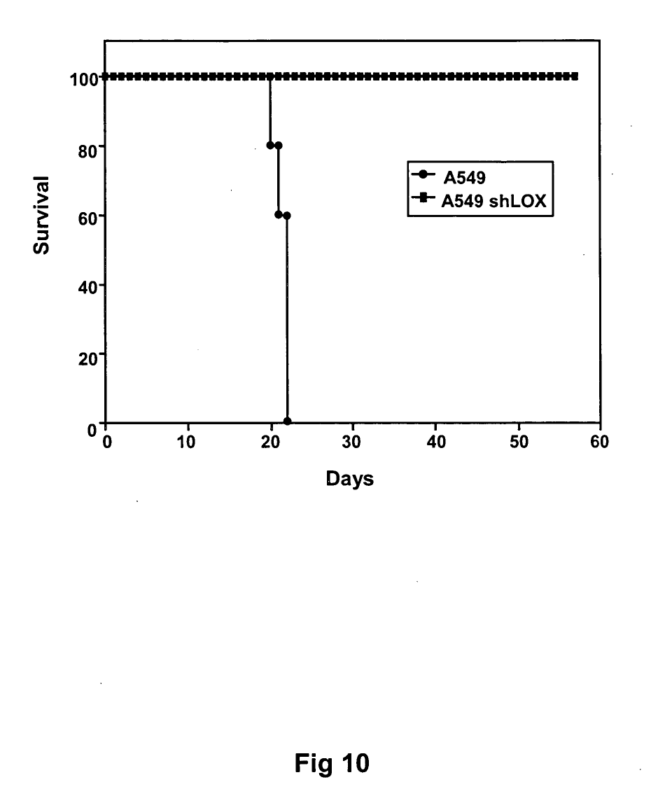

Human lung cancer A549 cells (ATCC CCL 185), known to be highly metastatic, were generated to stably express shRNA targeting LOX, as described in Methods. RT-PCR analysis revealed decreased LOX expression, particularly in hypoxic (H) conditions (versus N=normoxia). 1×10⁶ cells were injected into the tail vein of mice (all cells go to the lung). As can be seen in FIG. 10, all mice with control cells died within 22 days (N=5). Mice with cells expression the LOX shRNA lived beyond the experimental time shown (N=10).

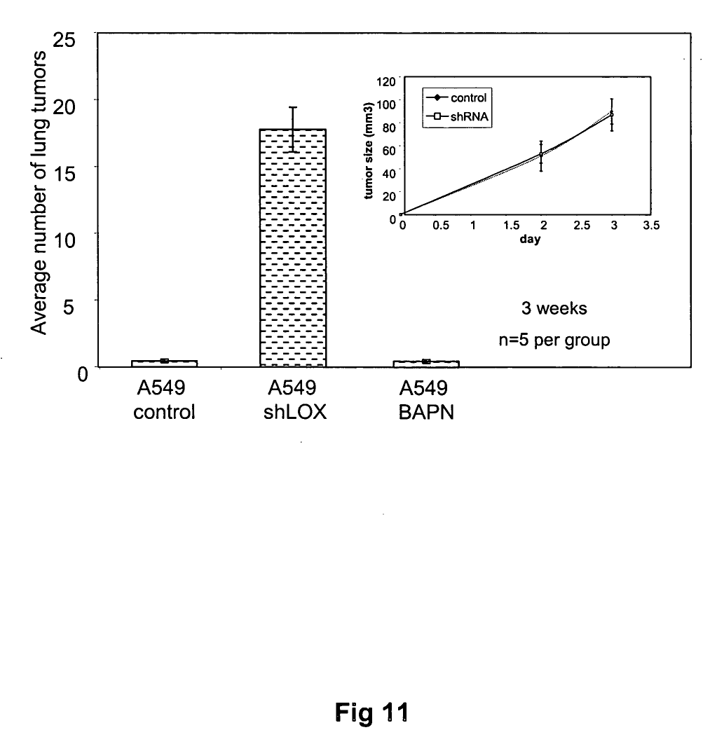

Lungs from the dead mice bearing control tumors were formalin fixed, paraffin embedded and H&E stained to quantify lung tumors. Most lungs contained no tumors but all mice had numerous lymph node metastases and one had liver metastases. N=5 mice bearing lung tumors expressing LOX shRNA were sacrificed at Day 22. These mice had numerous tumors in the lung but no metastases. In addition, n=5 mice bearing control tumors treated daily with BAPN (100 mg/kg) were sacrificed at Day 22. These mice had very few tumors and no metastases. The number of lung tumors for the A549 control animals is low because it is believed that the tumors metastasized out of the lung. The number of tumors in the BAPN treated group is low because the BAPN resulted in tumor cell death, showing an effect of LOX inhibition on primary tumor growth as well as metastatic growth. The inset of FIG. 11 shows growth rates of control and shRNA cells grown as subcutaneous tumors (1×10⁶ cells at day 0), which were the same for both cell lines. FIG. 11 shows that the number of lung tumors in shLOX tumors is seen to be significantly higher than in untreated tumors or tumors treated with BAPN.

### Example 13

### LOXL-2 and LOXL-4 Proteins are also Implicated in Cell Invasion and Metastisis

In another experiment, the expression levels of LOXL-1, LOXL-2, LOXL-3 and LOX-4 was examined under normal and hypoxic conditions by RT PCR analysis and imaging of blots (data not shown). As BAPN can also inhibit lysyl oxidase-like proteins, (LOXL 1-4), we investigated their expression levels and hypoxia responsiveness in MDA 231 human breast cancer cells and SiHa human cervical cancer cells. RT-PCR analysis revealed low expression of LOXLs 1+2, high expression of LOXL-3, and moderate expression of LOXL-4. None of the LOXL proteins showed hypoxic induction.

An in vitro invasion assay was carried out as described in Methods and as described in connection with FIG. 6. LOXL-1, 2, 3 and 4 were studied under normal and hypoxic conditions. It was found that hypoxic conditions increased invasiveness of wild type cells treated with LOX antisense. Also, in shLOXL-2 and shLOXL-4 cells, i.e., those having a vector against LOXL-2 or LOXL-4, no difference was seen compared to wild type LOXL-2 or LOXL-4 cells.

Scratch assays were performed to examine cell migration of LOXL-1, LOXL-2, LOXL-3, and LOXL-4 antisense-treated cells. As examined microscopically, only shLOXL-4 showed a slight defect in wound repair. Also, in an orthotopic tumor model of breast cancer, treatment with shRNA against LOXL-2 and LOXL-4 prevented formation of metastases.

LOXL-2 and LOXL-4 thus play a role which appears to be similar to that of LOX in tumor growth and metastasis.

### Example 14

### Mouse Monoclonal Antibody to LOX

The peptide given in Methods, which was used to immunize a rabbit, is used to immunize mice. BALB/c mice are injected with 160 mg of purified peptide. For the initial injection, the peptide is mixed with Freund's complete adjuvant (1:1) and injected subcutaneously. Subsequent injections are intraperitoneally in the absence of adjuvant. Serum antibody to LOX is determined by an enzyme linked immunosorbent assay (ELISA) in which the full length LOX protein is bound to polystyrene plates. After nine immunizations over a period of seven months, the spleen of one mouse with a high titer antibody directed against LOX is removed and fused with cells of the P₃ U₁ mouse plasmacytoma cell line. The resulting clones are screened for their ability to bind LOX and LOX catalytic domain in ELISA assays. A hybridoma-producing antibody reactive with LOX catalytic domain is isolated and subcloned. This hybridoma is grown in tissue culture media as well as in ascites to serve as a source of LOX antibody.

### Example 15

### Human Monoclonal Antibody to LOX

As described in EP 0239400 (Winter et al.), the above-described mouse monoclonal is altered by substitution of its complementarity determining regions (CDRs) into a human monoclonal antibody or monoclonal antibody fragment. The CDRs from human heavy and light chain Ig variable region domains with alternative CDRs from murine variable region domains. These altered Ig variable regions may subsequently be combined with human Ig constant regions to created antibodies, which are totally human in composition except for the substituted murine CDRs. Such CDR-substituted antibodies would be predicted to be less likely to elicit an immune response in humans compared to chimeric antibodies because the CDR-substituted antibodies contain considerably less non-human components. The process for humanizing monoclonal antibodies via CDR “grafting” has been termed “reshaping.” (Riechmann et al., 1988 Nature 332: 323-327, “Reshaping human antibodies for therapy”; Verhoeyen et al., 1988, Science 239: 1534-1536, “Reshaping of human antibodies using CDR-grafting in Monoclonal Antibodies”.

Transplantation of the murine LOX antibody CDRs is achieved by genetic engineering whereby CDR DNA sequences are determined by cloning of murine heavy and light chain variable (V) region gene segments, and are then transferred to corresponding human V regions by site directed mutagenesis. In the final stage of the process, human constant region gene segments of the desired isotype (usually gamma I for CH and kappa for CL) are added and the humanized heavy and light chain genes are co-expressed in mammalian cells to produce soluble humanized antibody.

The transfer of these CDRs to a human antibody confers on this antibody the antigen binding properties of the original murine antibody. The six CDRs in the murine antibody are mounted structurally on a V region “framework” region. The reason that CDR-grafting is successful is that framework regions between mouse and human antibodies may have very similar 3-D structures with similar points of attachment for CDRS, such that CDRs can be interchanged. Such humanized antibody homologs may be prepared, as exemplified in Jones et al., 1986 Nature 321: 522-525, “Replacing the complementarity-determining regions in a human antibody with those from a mouse”; Riechmann, 1988, Nature 332:323-327, “Reshaping human antibodies for therapy”; Queen et al., 1989, Proc. Nat. Acad. Sci. USA 86:10029, “A humanized antibody that binds to the interleukin 2 receptor” and Orlandi et al., 1989, Proc. Natl. Acad. Sci. USA 86:3833 “Cloning Immunoglobulin variable domains for expression by the polymerase chain reaction.”

Nonetheless, certain amino acids within framework regions are thought to interact with CDRs and to influence overall antigen binding affinity. The direct transfer of CDRs from a murine antibody to produce a humanized antibody without any modifications of the human V region frameworks often results in a partial or complete loss of binding affinity. Thus it may be desired to alter residues in the framework regions of the acceptor antibody in order to obtain binding activity.

Queen et al., 1989, Proc. Nat. Acad. Sci. USA 86: 10029-10033, “A humanized antibody that binds to the interleukin 2 receptor” and WO 90/07861 (Protein Design Labs Inc.) have described the preparation of a humanized antibody that contains modified residues in the framework regions of the acceptor antibody by combining the CDRs of a murine mAb (anti-Tac) with human immunoglobulin framework and constant regions. They have demonstrated one solution to the problem of the loss of binding affinity that often results from direct CDR transfer without any modifications of the human V region framework residues; their solution involves two key steps. First, the human V framework regions are chosen by computer analysts for optimal protein sequence homology to the V region framework of the original murine antibody, in this case, the anti-Tac MAb. In the second step, the tertiary structure of the murine V region is modeled by computer in order to visualize framework amino acid residues which are likely to interact with the murine CDRs and these murine amino acid residues are then superimposed on the homologous human framework. Their approach of employing homologous human frameworks with putative murine contact residues resulted in humanized antibodies with similar binding affinities to the original murine antibody with respect to antibodies specific for the interleukin 2 receptor (Queen et al., 1989 [supra]) and also for antibodies specific for herpes simplex virus (HSV) (Co. et al., 1991, Proc. Nat. Acad. Sci. USA 88: 2869-2873, “Humanised antibodies for antiviral therapy”.

Further details of this humanization procedure are given in U.S. Pat. No. 4,816,567 to Winter et al., U.S. Pat. No. 4,816,397 to Boss et al and U.S. Pat. No. 4,816,567 and U.S. Pat. No. 4,816,567 to Cabilly et al., all of which are known to those in the art and are specifically incorporated by reference for purposes of describing the exemplified preparation.

### Example 16

### LOX Inhibition Prevents Metastatic Tumor Growth in a Number of Different Cell Types

Described briefly below are the results of experiments conducted with several different cell types.
1. PANCREATIC: Panc-1 human pancreatic cells (see ATCC CRL-1469) expressing LOX shRNA were subjected to a tail vein metastasis assay comparing their metastatic growth to that of control Panc-1 cells. The same effect as with breast cancer cells was observed: mice injected with LOX shRNA expressing cells had fewer lung foci (micro-metastases).
2. CERVICAL: Tail vein metastasis assays were performed using Caski human cervical cancer cells (See CRL-1550). After 4 weeks of growth, half the mice were treated with BAPN. This eliminated formation of any lung foci (micro-metastases).
3. COLON: HCT116 human colon cancer cells (see CCL-247) were transfected to express LOX shRNA and grown as subcutaneous tumors in nude mice, while comparing their growth with that of control cells. Both tumor types grew at the same rate. However, within 4 weeks, all the mice bearing control tumors died and displayed widespread metastases particularly in the GI tract. In contrast, the mice bearing LOX shRNA expressing tumors survived until the experiment was terminated when the tumors reached the maximum size permitted by the protocol (1000 mm³). These mice displayed no metastases.

In addition, it should be noted that, as shown in FIG. 7, LOX inhibition has been shown here to prevent in vitro invasion of hypoxic cells of several other cancer types, namely, Head & Neck, Melanoma, Colon, Pancreatic, Lung and Renal.

### IV. Methods

Cell Culture and Oxygen Deprivation

Cell lines obtained from ATCC were routinely cultured and oxygen deprived for 18 h as previously described⁴⁷. DsRed SiHa cells were a gift from N. Dornhofer. Actinomycin D (5 μgml⁻¹), desferoxamine/CoCl₂/bathocuproinedisulphonic acid/catalase (100 mM) and BAPN (200 mM) were used at given concentrations (Sigma).

Retrospective Clinical Study

The average hypoxia score in each breast cancer sample was calculated by averaging the expression value of all 122 unique unigene clusters comprising the hypoxia gene signature without LOX, as described previously. The correlation between the averaged hypoxia score and LOX expression in each breast cancer sample was then calculated. For Kaplan-Meyer plots, LOX expression levels were determined as described, plotting top and bottom tiers.

Immunology Studies

Methods were performed as previously described. The primary antibodies used were HIF-1α (BD Transduction Labs), alpha-tubulin (Research Diagnostics), and phoshpho-FAK (Santa Cruz). LOX was analyzed by using a rabbit polyclonal antibody raised against a synthetic peptide of human LOX (EDTSCDYGYHRRFA (SEQ ID NO: 1); Open Biosystems). Hypoxic regions were identified by staining for Pimonidazole. For LOX immunoblotting, proteins in the conditioned media were concentrated using Microcon filters (Millipore), 20 μl samples were loaded. Quantification was performed with ImageQuant software.

Semiguantitative and Fully Quantitative RT-PCR Analysis

Methods were performed as previously described. The Taqman PCR primer sequences for LOX were as follows: ATGAGTTTAGCCACTTGTACCTGCTT SEQ ID NO:2) and AAACTTGCTTTGTGGCCTTCA SEQ ID NO: 3).

Hypoxia Responsiveness of the LOX Promoter

The luciferase assay was performed as previously described. The LOX promoter sequence was mutated by site directed mutagenesis (Stratagene), according to the manufacturer's instructions. A positive control containing 5 hypoxia responsive elements (HREs) was additionally used.

Transient and Retroviral Transfection of Cells

Treatment of cells with sense or antisense phosphorothioate-modified LOX-specific sense or antisense oligonucleotides (Integrated DNA Technologies) was performed as previously described (ref. 29). HIF-1 oxygen-dependent degradation (ODD) domain and proline double mutant constructs were a gift of D. Chan. Mature LOX and the LOX delta cat mutant (missing catalytic domain) were gifts from A. Di Donato. The HIF-1α shRNA construct is described in Erler et al. “Hypoxia-Mediated Down-Regulation of Bid and Bax in Tumors Occurs via Hypoxia-Inducible Factor 1-Dependent and -Independent Mechanisms and Contributes to Drug Resistance,” Molecular and Cellular Biology, April 2004, p. 2875-2889, Vol. 24, No. 7. All transient transfections were carried out using Lipofectamine 2000 (Invitrogen) according to the manufacturer's instructions. Transfection efficiencies were determined for each cell line via co-transfection with a GFP-expressing vector, which were approximately: 90% for, and 50% for MDA 231 and 435 cells.

MDA-MB 231 and SiHa human cancer cells were retrovirally transfected with a pBabe vector, containing a LOX-specific targeting sequence (5′-GTTCCTGCTCTCAGTAACC-3′ SEQ ID NO: 4)). This sequence was checked against the database to confirm specificity. As a negative control, a scrambled sequence was used (5′-CACATGTTCCGATCTCGGC-3′ SEQ ID NO: 5)). Infected cells were selected in puromycin (Sigma) for several weeks and then polyclonal cell populations tested for reduced LOX expression levels. The pBABE vector is further described at Spicher et al. Ref. 49.

In Vitro Invasion Analysis

Invasive behavior was examined in vitro as previously described. Briefly, cells were serum deprived for 24 h then 2.5×10⁴ cells were seeded in triplicate on both Matrigel-coated and uncoated inserts, moved to chambers containing 750 μl of FBS as a chemo-attractant and incubated at in normoxic or oxygen deprived conditions for 24 hr. Treatments with beta-aminoproprionitrile or bathocuprione disulphonate (Sigma) were performed 24 h prior to serum deprivation, and continued throughout the experiment.

Assessment of adhesion-free and 3D growth of cells in culture. For monolayer growth curves, cells were plated and counted each day using a hemocytometer. 3D growth in Matrigel and type I collagen (both BD Biosciences) was performed as previously described. Briefly, 2×10⁴ cells were seeded and grown for three days prior to four-day incubation (collagen), or simply for 10 days (matrigel) at normoxia or hypoxia.

In Vivo Tail Vein Metastasis Assay

MDA 231 cells expressing either LOX siRNA or a scrambled control sequence were injected intravenously with 5×10⁵ cells in 0.1 ml of DMEM via the tail vein. A total of ten mice were injected per cell line. Four weeks after injection, mice were euthanized. Microscopic quantitation of lung foci was performed on representative cross-sections of formalin-fixed, paraffin-embedded lungs stained with hematoxylin and eosin. Correct identification of micro-metastases (minimum of four human cells with large nuclei) was kindly confirmed by a board-certified veterinary pathologist.

Growth of MDA-MB 231 Cells as Orthotopic Tumors

MDA-MB 231 cells were grown as subcutaneous orthotopic tumors in 6-week-old female Nude (nu/nu) mice following intradermal injection of 1×10⁷ cells in 0.1 ml of PBS into the mammary fat pad. Mice were sacrificed and tumors excised six weeks after inoculation. Some mice were treated for twice weekly with up to 20 mg kg⁻¹ purified LOX antibody, two weeks after inoculation, or daily with 100 mg kg⁻¹ BAPN (intraperitoneally), for the last four, three or two weeks of tumor growth (4, 3 and 2 wk BAPN, respectively; these doses and durations have no deleterious side effects). Lung and liver metastases were defined as gross lesions of at least 25 cells. The presence of human tumor cells was verified by cytokeratin staining (not shown).

LOX activity assay. The original method is described in Folgelgren, et al. J. Biol. Chem. 280:24690-24697 (2005). For the in vivo data, terminal bleeds were taken at the end of the experiment described above from untreated (control) mice, 2 and 3 wk BAPN mice, and antibody-treated mice. Plasma (10 ml) was tested and fluorescence (a measure of LOX activity) was plotted, where 0=sample p 500 μM BAPN (complete LOX inhibition). For the in vitro data, 50 μl of conditioned phenol-red-free medium was taken from cells incubated for 24 h under conditions of hypoxia. Samples were incubated overnight at 37° C. with different concentrations of BAPN or with purified LOX antibody at a concentration equivalent to 20 mg kg⁻¹ dose. Again, 0=fluorescent reading for 500 μM BAPN. No activity could be detected in CM from aerobic and LOX shRNA-expressing cells, or in blood from mice that did not bear tumors (data not shown).

Adhesion Assay

For MDA231 cells: 2.5×10⁵ cells were plated in serum-free media in collagen-coated 96-well plates. Adherent cells were trypsinized and counted using a hemocytometer over a time course of 8 h. For DSRED SiHa cells: 2.5×10⁵ cells were plated in serum-free media and left to adhere. Wells were washed and media replaced with PBS at specific times over an 8 h time course, after which fluorescence was measured and the number of adherent cells determined from the standard curve (generated with 2.5×10³ to 5×10⁵ cells).

### V. Summary and Conclusion

Several factors might explain why LOX is required for metastatic growth. First, LOX is required for FAK activity, which is known to mediate cell proliferation and survival. Second, LOX might regulate FN activity through FAK activation, providing a permissive niche to support metastatic tumor cell growth. Last, LOX activity is essential for the formation of a mature ECM, which is undoubtedly required for survival signaling and cellular growth. It is noteworthy that we did not observe significant effects of LOX inhibition on primary tumor growth, whereas we found marked effects on metastatic growth in the lungs and liver. In particular, shRNA expressing cells orthotopically implanted grew as primary tumors with the same kinetics as wild-type cells. These data indicate that the effects of LOX on cell adhesion, migration, invasion and three-dimensional growth are less crucial for primary growth than for metastatic growth.

The data presented here provide strong evidence that LOX is a good therapeutic target for the prevention of metastasis in breast cancer, and that targeting secreted LOX presents a mechanism for preventing early and late stages of metastasis. It is shown here that hypoxia increases LOX mRNA, LOX protein, and secreted LOX activity, resulting in enhanced invasive migration required for metastatic spread. In addition, the remodeled matrix tracks resulting from increased LOX activity and cell migration could provide a highway along which other cells can travel more easily, thus increasing migration and invasion. Although LOX is known to be induced and/or activated by growth factors such as transforming growth factor-β, hypoxia might be more clinically relevant with regard to tumor progression. Many studies have shown that hypoxia promotes the aggressiveness of cancer cells. Whereas inhibition of LOX under aerobic conditions affected tumor cell invasion only modestly, inhibition of the much higher LOX expression levels observed under hypoxia consistently produced more marked effects. Furthermore, the aerobic conditions (21% O₂) typically employed for many in vitro assays are actually hyperoxic relative to oxygenation levels experienced in the body, particularly within solid tumors. Indeed, LOX expression was undetectable in some tumor cell lines without oxygen deprivation (FIG. 1), again highlighting the relevance and clinical implications of hypoxia-induced levels of LOX.

Hypoxia-induced LOX has a key function in tumor metastasis. The data discussed here provide mechanistic evidence for hypoxia-driven metastasis and the influence of the ECM on metastatic spread, and support the therapeutic targeting of LOX to prevent and treat metastatic disease. The data provided here also demonstrate that human hypoxic cancer cells have enhanced invasive and metastatic potentials in vitro and in vivo through increased migration and survival in hostile environments. These effects can be blocked by inhibition of LOX expression. Although LOX expression is known to be induced by other agents (such as TGF-β), hypoxia is the most clinically relevant in tumor progression. Hypoxia increased LOX mRNA levels in the tumor cells through a HIF-1 driven increase in transcript expression and stability, via an HRE in the LOX promoter. This resulted in enhanced LOX pro-enzyme secretion out of the cell where it is processed, increasing the amount of active, mature enzyme in the ECM. LOX acts on collagen fibers in the stroma surrounding the tumor cells, increasing ECM deposition and fibrosis (aberrant collagen deposition), and LOX additionally interacts with fibronectin. As fibrillar collagen and fibronectin are both major ligands for integrins, the data suggests that LOX activity results in increased integrin stimulation enhancing the focal adhesion formation, FAK activation and cell motility observed in hypoxia that were all LOX-dependent. The remodeled matrix tracks resulting from increased LOX activity and cell migration would additionally provide a “road” along which other cells could travel more easily, increasing migration and metastasis. In agreement with our findings, transition from a localized to invasive or metastatic phenotype in some cancer types (such as breast cancer) is often associated with the formation of fibrotic foci and desmoplasia (the presence of unusually dense collagenous stroma). Once the tumor has achieved the dedifferentiated stage of single-cell dissemination such as during EMT as observed in our hypoxic cells, metastatic spread is increased, resulting in poor prognosis. Indeed, we found LOX expression levels to be clinically related to metastasis-free and overall survival in head and neck, and breast cancer patients. In breast cancer, LOX expression was associated with decreased survival in ER negative (but not ER positive) breast cancer patients. Patients with this tumor type are known to have a worse prognosis and display very aggressive tumors.

The data presented herein provide strong evidence that LOX is a good therapeutic target for the prevention of metastasis in breast cancer. Targeting secreted LOX presents an attractive mechanism to prevent all stages of metastasis. Inhibition of LOX blocks invasion and metastasis of hypoxic breast cancer cells and reduces these effects in aerobic cells. The data demonstrated that these effects were due to the critical role LOX plays in cell-matrix adhesion and in ECM survival signaling, which have not previously been reported. By inhibiting LOX expression and/or activity, cells were unable to move (even passively) preventing cell migration and invasion of adjacent tissues, and cells were additionally unable to survive well in hostile environments such as the vascular system or a new host environment. The data indicated that this may be due to decreased proliferation and increased apoptosis caused by interference with cell-matrix adhesion interactions. We have used a purified antibody specific to LOX to produce similar results in mice relative to that observed with BAPN treatment, but without deleterious side-effects to collagen-rich normal tissues.

The data provided mechanistic evidence for hypoxia-driven metastasis, and the influence of the ECM on metastasis, and supported targeting hypoxia-driven pathways to prevent and treat metastatic breast cancer and potentially other solid tumors, by targeting LOX. This is the first report, to our knowledge, where inhibition of a hypoxia-related protein completely preventing metastasis in a breast cancer model.

The present examples, methods, procedures, specific compounds and molecules are meant to exemplify and illustrate the invention and should in no way be seen as limiting the scope of the invention, which is defined by the literal and equivalent scope of the appended claims. Any patents or publications mentioned in this specification are indicative of levels of those skilled in the art to which the patent pertains and are intended to convey details of the invention which may not be explicitly set out but would be understood by workers in the field. Such patents or publications are hereby incorporated by reference to the same extent as if each was specifically and individually incorporated by reference and for the purpose of describing and enabling the method or material referred to.

### V. References

1. Hockel, M. & Vaupel, P. Tumor hypoxia: definitions and current clinical, biologic, and molecular aspects. J Natl Cancer Inst 93, 266-76 (2001).
2. Subarsky, P. & Hill, R. P. The hypoxic tumour microenvironment and metastatic progression. Clin Exp Metastasis 20, 237-50 (2003).
3. Taghian, A. G. et al. Paclitaxel decreases the interstitial fluid pressure and improves oxygenation in breast cancers in patients treated with neoadjuvant chemotherapy: clinical implications. J Clin Oncol. 23(9),1951-61 (2005).
4. Steeg, P. S. Molecular biology of breast cancer metastasis. ‘Has it spread?’: disarming one of the most terrifying questions. Breast Cancer Res. 2(6), 396-9 (2000).
5. Chang, C. & Werb, Z. The many faces of metalloproteases: cell growth, invasion, angiogenesis and metastasis. Trends Cell Biol 11, S37-43 (2001).
6. Radisky, D., Hagios, C. & Bissell, M. J. Tumors are unique organs defined by abnormal signaling and context. Semin Cancer Biol 11, 87-95 (2001).
7. Brody, J. S., Kagan, H. & Manalo, A. Lung lysyl oxidase activity: relation to lung growth. Am Rev Respir Dis 120, 1289-95 (1979).
8. Denko, N. C. et al. Investigating hypoxic tumor physiology through gene expression patterns. Oncogene 22, 5907-14 (2003).
9. Kagan, H. M. & Li, W. Lysyl oxidase: properties, specificity, and biological roles inside and outside of the cell. J Cell Biochem 88, 660-72 (2003).
10. Hornstra, I. K. et al. Lysyl oxidase is required for vascular and diaphragmatic development in mice. J Biol Chem 278, 14387-93 (2003).
11. Pinnell, S. R. Molecular defects in the Ehlers-Danlos syndrome. J Invest Dermatol 79 Suppl 1, 90s-92s (1982).
12. Royce, P. M., Camakaris, J. & Danks, D. M. Reduced lysyl oxidase activity in skin fibroblasts from patients with Menkes' syndrome. Biochem J 192, 579-86 (1980).
13. Khakoo, A. et al. Congenital cutis laxa and lysyl oxidase deficiency. Clin Genet 51, 109-14 (1997).
14. Kagan, H. M. Lysyl oxidase: mechanism, regulation and relationship to liver fibrosis. Pathol Res Pract 190, 910-9 (1994).
15. Chanoki, M. et al. Increased expression of lysyl oxidase in skin with scleroderma. Br J Dermatol 133, 710-5 (1995).
16. Ooshima, A. & Midorikawa, O. Increased lysyl oxidase activity in blood vessels of hypertensive rats and effect of beta-aminopropionitrile on arteriosclerosis. Jpn Circ J 41, 1337-40 (1977).
17. Csiszar, K. Lysyl oxidases: a novel multifunctional amine oxidase family. Prog Nucleic Acid Res Mol Biol 70, 1-32 (2001).
18. Li, W. et al. Localization and activity of lysyl oxidase within nuclei of fibrogenic cells. Proc Natl Acad Sci USA 94, 12817-22 (1997).
19. Giampuzzi, M. et al. Lysyl oxidase activates the transcription activity of human collagen III promoter. Possible involvement of Ku antigen. J Biol Chem 275, 36341-9 (2000).
20. Lazarus, H. M., Cruikshank, W. W., Narasimhan, N., Kagan, H. M. & Center, D. M. Induction of human monocyte motility by lysyl oxidase. Matrix Biol 14, 727-31 (1995).
21. Nelson, J. M., Diegelmann, R. F. & Cohen, I. K. Effect of beta-aminopropionitrile and ascorbate on fibroblast migration. Proc Soc Exp Biol Med 188, 346-52 (1988).
22. Li, W., Liu, G., Chou, I. N. & Kagan, H. M. Hydrogen peroxide-mediated, lysyl oxidase-dependent chemotaxis of vascular smooth muscle cells. J Cell Biochem 78, 550-7 (2000).
23. Smith-Mungo, L. I. & Kagan, H. M. Lysyl oxidase: properties, regulation and multiple functions in biology. Matrix Biol 16, 387-98 (1998).
24. Ren, C., Yang, G., Timme, T. L., Wheeler, T. M. & Thompson, T. C. Reduced lysyl oxidase messenger RNA levels in experimental and human prostate cancer. Cancer Res 58, 1285-90 (1998).
25. Csiszar, K. et al. Somatic mutations of the lysyl oxidase gene on chromosome 5q23.1 in colorectal tumors. Int J Cancer 97, 636-42 (2002).
26. Kaneda, A. et al. Lysyl oxidase is a tumor suppressor gene inactivated by methylation and loss of heterozygosity in human gastric cancers. Cancer Res 64, 6410-5 (2004).
27. Stassar, M. J. et al. Identification of human renal cell carcinoma associated genes by suppression subtractive hybridization. Br J Cancer 85, 1372-82 (2001).
28. Kirschmann, D. A., Seftor, E. A., Nieva, D. R., Mariano, E. A. & Hendrix, M. J. Differentially expressed genes associated with the metastatic phenotype in breast cancer. Breast Cancer Res Treat 55, 127-36 (1999).
29. Kirschmann, D. A. et al. A molecular role for lysyl oxidase in breast cancer invasion. Cancer Res 62, 4478-83 (2002).
30. Decitre, M. et al. Lysyl oxidase-like protein localizes to sites of de novo fibrinogenesis in fibrosis and in the early stromal reaction of ductal breast carcinomas. Lab Invest 78, 143-51 (1998).
31. Colpaert, C. G. et al. The presence of a fibrotic focus in invasive breast carcinoma correlates with the expression of carbonic anhydrase IX and is a marker of hypoxia and poor prognosis. Breast Cancer Res Treat 81, 137-47 (2003).
32. Chan, D. A., Sutphin, P. D., Denko, N. C. & Giaccia, A. J. Role of prolyl hydroxylation in oncogenically stabilized hypoxia-inducible factor-1alpha. J Biol Chem 277, 40112-7 (2002).
33. Csiszar, K., Entersz, I., Trackman, P. C., Samid, D. & Boyd, C. D. Functional analysis of the promoter and first intron of the human lysyl oxidase gene. Mol Biol Rep 23, 97-108 (1996).
34. Liu, L. X. et al. Stabilization of vascular endothelial growth factor mRNA by hypoxia-inducible factor 1. Biochem Biophys Res Commun 291, 908-14 (2002).
35. Gacheru, S. N. et al. Transcriptional and post-transcriptional control of lysyl oxidase expression in vascular smooth muscle cells: effects of TGF-beta 1 and serum deprivation. J Cell Biochem 65, 395-407 (1997).
36. Cen, D., Brayton, D., Shahandeh, B., Meyskens, F. L., Jr. & Farmer, P. J. Disulfiram facilitates intracellular Cu uptake and induces apoptosis in human melanoma cells. J Med Chem 47, 6914-20 (2004).
37. Janda, E. et al. Ras and TGF[beta] cooperatively regulate epithelial cell plasticity and metastasis: dissection of Ras signaling pathways. J Cell Biol 156, 299-313 (2002).
38. Luedeke, J. D., McCall, R. D., Dillaman, R. M. & Kinsey, S. T. Properties of slow- and fast-twitch skeletal muscle from mice with an inherited capacity for hypoxic exercise. Comp Biochem Physiol A Mol Integr Physiol 138, 373-82 (2004).
39. Gilad, G. M. & Gilad, V. H. Beta-aminopropionitrile treatment can accelerate recovery of mice after spinal cord injury. Eur J Pharmacol 430, 69-72 (2001).
40. Chia, S. K. et al. Prognostic significance of a novel hypoxia-regulated marker, carbonic anhydrase IX, in invasive breast carcinoma. J Clin Oncol 19, 3660-8 (2001).
41. Chang, H. Y. et al. Robustness, scalability, and integration of a wound-response gene expression signature in predicting breast cancer survival. Proc Natl Acad Sci USA 102, 3738-43 (2005).
42. Hasebe, T., Mukai, K., Tsuda, H. & Ochiai, A. New prognostic histological parameter of invasive ductal carcinoma of the breast: clinicopathological significance of fibrotic focus. Pathol Int 50, 263-72 (2000).
43. Friedman, S. L. Molecular regulation of hepatic fibrosis, an integrated cellular response to tissue injury. J Biol Chem 275, 2247-50 (2000).
44. Mott, J. D. & Werb, Z. Regulation of matrix biology by matrix metalloproteinases. Curr Opin Cell Biol 16, 558-64 (2004).
45. Kassis, J., Lauffenburger, D. A., Turner, T. & Wells, A. Tumor invasion as dysregulated cell motility. Semin Cancer Biol 11, 105-17 (2001).
46. Dyson, S. J. Medical management of superficial digital flexor tendonitis: a comparative study in 219 horses (1992-2000). Equine Vet J36, 415-9 (2004).
47. Erler, J. T. et al. Hypoxia-mediated down-regulation of Bid and Bax in tumors occurs via hypoxia-inducible factor 1-dependent and -independent mechanisms and contributes to drug resistance. Mol Cell Biol 24, 2875-89 (2004).
48. Tazuke, S. I. et al. Hypoxia stimulates insulin-like growth factor binding protein 1 (IGFBP-1) gene expression in HepG2 cells: a possible model for IGFBP-1 expression in fetal hypoxia. Proc Natl Acad Sci USA 95, 10188-93 (1998).
49. Spicher, A. et al. Highly conserved RNA sequences that are sensors of environmental stress. Mol Cell Biol 18, 7371-82 (1998).
50. Bedogni, B. et al. Topical treatment with inhibitors of the phosphatidylinositol 3′-kinase/Akt and Raf/mitogen-activated protein kinase kinase/extracellular signal-regulated kinase pathways reduces melanoma development in severe combined immunodeficient mice. Cancer Res 64, 2552-60 (2004).
51. Chen Y., et al., Cancer Res 64, 7302 (2004).
52. Swinson D E, et al., J Clin Oncol 21:473-82 (2003).

What is claimed is:

1. A method of reducing tumor growth, comprising
administering to a subject in need thereof an effective amount of an inhibitor of lysyl oxidase activity; and
optionally, a pharmaceutically acceptable carrier.

2. The method of claim 1 wherein the tumor growth is metastatic tumor growth.

3. The method of claim 1, wherein the inhibitor is an antibody, a small molecule inhibitor, siRNA, shRNA or an antisense polynucleotide.

4. The method of claim 1, wherein the inhibitor is a small molecule inhibitor.

5. The method of claim 4 where the small molecule inhibitor is a pro-drug activated under hypoxic conditions.

6. The method of claim 1 wherein the inhibitor is an antibody.

7. The method of claim 6 wherein the antibody is administered intravenously.

8. The method of claim 6 wherein the antibody is a human antibody.

9. The method of claim 1 wherein the inhibitor is a nucleic acid targeting lysyl oxidase mRNA.

10. The method of claim 9, wherein the inhibitor is shRNA.

11. The method of claim 1 where the tumor is a solid tumor.

12. The method of 11 wherein the tumor is selected from the group consisting of a breast tumor, a pancreas tumor, a lung tumor, a cervical tumor, a colon tumor and a head and neck tumor.

13. The method of claim 12 wherein the tumor growth is metastatic tumor growth.

14. The method of claim 13 wherein the inhibitor is a small molecule inhibitor of lysyl oxidase.

15. The method of claim 13 where the inhibitor is selected from the group consisting of an antibody, shRNA, siRNA, and an antisense polynucleotide.

16. The method of claim 1 further comprising the step of administering to the subject an anticancer agent.

17. The method of claim 1 wherein the inhibitor specifically inhibits a lysyl oxidase selected from the group consisting of LOX, LOXL-2 and LOXL-4.

18. The method of claim 1 wherein the inhibitor is administered to a subject with an ER negative breast tumor.

19. A method for identifying a compound that inhibits tumor cell growth, comprising:
contacting lysyl oxidase with a candidate compound;
determining the activity of the lysyl oxidase when contacted with the candidate compound; and
determining the growth of a tumor when treated with the candidate compound,
wherein the candidate compound that reduces the activity of said lysyl oxidase compared to the activity detected in the absence of the compound is identified as the compound that inhibits tumor cell growth.

20. The method of claim 19, wherein the candidate compound is contacted with the metastatic cell under hypoxic conditions.

21. The method of claim 19, wherein the candidate compound is a peptide, an antibody, a small molecule inhibitor, siRNA, shRNA, or an antisense polynucleotide.

22. The method of claim 19, wherein the cell is contacted in vivo for determining tumor growth inhibition.

23. The method of claim 19 where the step of determining the growth of a tumor comprises measuring phospho-FAK with and without the candidate compound, whereby a LOX inhibitor is further identified by a reduction in phospho-FAK.

24. A method to stage tumor growth in a cancer patient, comprising assessing lysyl oxidase level in the blood, whereby lysyl oxidase level indicates a risk of having metastatic tumor growth.

25. The method of claim 24 wherein assessing lysyl oxidase activity comprises measurement of lysyl oxidase protein.

26. The method of claim 24 wherein assessing lysyl oxidase activity comprises measurement of lysyl oxidase enzymatic activity on a substrate.

27. The method of claim 24, wherein the lysyl oxidase activity is determined by measuring amounts of lysyl oxidase protein in the patient's blood.

28. The method of claim 27 wherein the amount of lysyl oxidase protein in the blood is measured with an antibody to lysyl oxidase.

29. The method of claim 24, wherein the lysyl oxidase activity is determined by measuring the level of enzymatic activity of lysyl oxidase from the patient on a substrate thereof.

30. The method of claim 24, wherein the lysyl oxidase is measured in a tumor sample taken from the patient.

31. The method of claim 24 wherein the lysyl oxidase activity is determined by measuring the level of a lysyl oxidase byproduct.

32. A method for inhibiting metastatic tumor growth in a subject having a primary tumor, comprising the step of administering to the subject an effective amount of an inhibitor of LOX enzyme.


SEQUENCE LISTING (7 sequences)

SEQ ID NO: 1 - protein, 14 aa, Artificial Sequence
  Description of Artificial Sequence Synthetic peptide
EDTSCDYGYHRRFA

SEQ ID NO: 2 - DNA, 26 bp, Artificial Sequence
  Description of Artificial Sequence Synthetic primer
atgagtttagccacttgtacctgctt

SEQ ID NO: 3 - DNA, 21 bp, Artificial Sequence
  Description of Artificial Sequence Synthetic primer
aaacttgctttgtggccttca

SEQ ID NO: 4 - DNA, 19 bp, Artificial Sequence
  Description of Artificial Sequence Synthetic oligonucleotide
gttcctgctctcagtaacc

SEQ ID NO: 5 - DNA, 19 bp, Artificial Sequence
  Description of Artificial Sequence Synthetic oligonucleotide
cacatgttccgatctcggc

SEQ ID NO: 6 - DNA, 20 bp, Artificial Sequence
  Description of Artificial Sequence Synthetic oligonucleotide
ccttccctcacgtgatttga

SEQ ID NO: 7 - DNA, 20 bp, Artificial Sequence
  Description of Artificial Sequence Synthetic oligonucleotide
ccttccctcttttgatttga